(19) United States
(12) Patent Application Publication
(10) Pub. No.: US 2021/0319864 A1
(43) Pub. Date: Oct. 14, 2021

(54) IDENTITY SYSTEMS THAT TRACK AND PERFORM ACTIONS USING HEALTH DATA

(71) Applicant: Alclear, LLC, New York, NY (US)

(72) Inventors: Rob Wisniewski, New York, NY (US); Marisa Bass, New York, NY (US); Catesby Perrin, New York, NY (US)

(21) Appl. No.: 17/226,391

(22) Filed: Apr. 9, 2021

Related U.S. Application Data
(60) Provisional application No. 63/008,319, filed on Apr. 10, 2020.
(60) Provisional application No. 63/013,378, filed on Apr. 21, 2020.
(60) Provisional application No. 63/053,014, filed on Jul. 17, 2020.

(51) Int. Cl. G16H 10/60; G16H 10/40; G16H 50/30; G16H 15/00; G06Q 10/02; G06F 21/62; A61B 5/01; A61B 5/00

(52) U.S. Cl. CPC G16H 10/60; G16H 10/40; G16H 50/30; G16H 15/00; G06Q 50/265; G06F 21/6245; A61B 5/01; A61B 5/7275; A61B 5/7282; G06Q 10/02

(57) ABSTRACT

A system tracks and performs actions using health data. The system is operable to use digital representations of biometrics to control access to identity information for people stored in an identification system. The system uses the stored identity information to track, evaluate, and/or correlate current and/or previously monitored health information for the people to perform one or more of a variety of actions. Such actions may include determining whether or not to allow the person access, providing attestations about a person's health information, routing the person based on one or more evaluations of the health information, and so on.

### CROSS-REFERENCE TO RELATED APPLICATION(S)

This application is a nonprovisional patent application of and claims the benefit of U.S. Provisional Patent Application No. 63/008,319, filed Apr. 10, 2020 and titled “Identity Systems that Track and Perform Actions Using Health Data,” U.S. Provisional Patent Application No. 63/013,378, filed Apr. 21, 2020 and titled “Identity Systems that Track and Perform Actions Using Health Data,” and U.S. Provisional Patent Application No. 63/053,014, filed Jul. 17, 2020 and titled “Identity Systems that Track and Perform Actions Using Health Data,” the disclosures of which are hereby incorporated herein by reference in their entireties.

### FIELD

The described embodiments relate generally to identity systems. More particularly, the present embodiments relate to identity systems that track and perform actions using health data.

### BACKGROUND

Communicable diseases are an unfortunate fact of life. Such communicable diseases include measles, mumps, influenza, swine flu, COVID-19, and many more. Vaccines may be developed to prevent the spread of such communicable diseases, but vaccines take time to develop, not everyone may be vaccinated, communicable diseases mutate, new communicable diseases emerge, and so on. Regardless of medical advancements, it is most likely that people will always have to deal with communicable diseases.

One way to deal with the fact of communicable diseases is to prevent contact between people with communicable diseases and vulnerable people, such as people who have not been vaccinated for the respective communicable disease, immunocompromised people, the very young, the very old, and so on. Typically, this involves either people who know they have communicable diseases voluntarily isolating themselves or medically trained personnel to observe and/or test for symptoms of communicable diseases.

### SUMMARY

The present disclosure relates to tracking and performing actions using health data. The system is operable to use digital representations of biometrics to control access to identity information for people stored in an identification system. The system uses the stored identity information to track, evaluate, and/or correlate current and/or previously monitored health information for the people to perform one or more of a variety of actions. Such actions may include determining whether or not to allow the person access, providing attestations about a person's health information, routing the person based on one or more evaluations of the health information, and so on.

In various embodiments, a system for tracking and performing actions using health data includes at least one non-transitory storage medium that stores instructions and at least one processor. The at least one processor executes the instructions to obtain a digital representation of a biometric for a person, determine an identity for the person using the digital representation of the biometric, determine an access account identifier stored in identity information associated with the identity, use the access account identifier to determine whether or not the person has an access permission, evaluate heath information for the person, and determine whether to allow the person access based on the access permission and the health information.

In some examples, the at least one processor obtains the health information for the person using a sensor. In a number of implementations of such examples, the at least one processor obtains the digital representation of the biometric for the person using the sensor.

In various examples, the at least one processor obtains the health information from a data store associated with the identity information. In some implementations of such examples, the health information includes a recently monitored temperature for the person.

In a number of examples, the health information includes a currently monitored temperature for the person. In various examples, the at least one processor is operable to receive the health information and store the health information in association with the identity information.

In some embodiments, a system for tracking and performing actions using health data includes at least one non-transitory storage medium that stores instructions and at least one processor. The at least one processor executes the instructions to obtain a digital representation of a biometric for a person, determine an identity for the person using the digital representation of the biometric, retrieve health information for the person stored in association with identity information associated with the identity, evaluate the health information, and provide an attestation based on the health information.

In various examples, the digital representation of the biometric is a first digital representation of the biometric and the health information includes data previously received with a second digital representation of the biometric by the at least one processor.

In a number of examples, the attestation indicates that the person has a particular vaccination or results of an antibody test evidence that the person has had a particular communicable illness and recovered. In some examples, the attestation indicates that a recently monitored temperature for the person is a normal temperature. In a number of examples, the attestation indicates that a recently monitored temperature for the person is an abnormal temperature. In various examples, the attestation indicates that the person is not a significant risk of having a particular communicable illness. In some examples, the at least one processor is operable to receive at least one medical record associated with the identity, verify the at least one medical record, and store the at least one medical record in association with the identity information.

In a number of embodiments, a system for tracking and performing actions using health data includes at least one non-transitory storage medium that stores instructions and at least one processor. The at least one processor executes the instructions to obtain a digital representation of a biometric for a person, determine an identity for the person using the digital representation of the biometric, evaluate health information for the person stored in association with identity information associated with the identity, and route the person based on the health information.

In some examples, the at least one processor routes the person by assigning the person a seat.

In various examples, the at least one processor evaluates the health information to determine that the person encountered an infected person. In some implementations of such examples, the at least one processor determines whether the person is tested for a communicable illness after encountering the infected person. In various implementations of such examples, the at least one processor determines whether the person is vaccinated for a communicable illness after encountering the infected person or that results of an antibody test evidence that the person has had the communicable illness and recovered.

In a number of examples, the at least one processor routes the person in a first manner if the person is not at risk of having a communicable illness and a second manner if the person cannot be determined to not be at risk of having the communicable illness.

### BRIEF DESCRIPTION OF THE DRAWINGS

The disclosure will be readily understood by the following detailed description in conjunction with the accompanying drawings, wherein like reference numerals designate like structural elements.

FIG. 1 depicts a first example system for tracking and performing actions using health data.

FIG. 2 is a flow chart illustrating a first example method for tracking and performing actions using health data. This method may be performed by the system of FIG. 1.

FIG. 3 is a flow chart illustrating a second example method for tracking and performing actions using health data. This method may be performed by the system of FIG. 1.

FIG. 4 is a flow chart illustrating a third example method for tracking and performing actions using health data. This method may be performed by the system of FIG. 1.

FIG. 5 is a flow chart illustrating a fourth example method for tracking and performing actions using health data. This method may be performed by the system of FIG. 1.

FIG. 6 is a flow chart illustrating a fifth example method for tracking and performing actions using health data. This method may be performed by the system of FIG. 1.

FIG. 7 is a flow chart illustrating a sixth example method for tracking and performing actions using health data. This method may be performed by the system of FIG. 1.

FIG. 8 is a flow chart illustrating a seventh example method for tracking and performing actions using health data. This method may be performed by the system of FIG. 1.

FIG. 9 is a flow chart illustrating an eighth example method for tracking and performing actions using health data. This method may be performed by the system of FIG. 1.

FIG. 10 is a flow chart illustrating a ninth example method for tracking and performing actions using health data. This method may be performed by the system of FIG. 1.

FIG. 11 is a flow chart illustrating a tenth example method for tracking and performing actions using health data. This method may be performed by the system of FIG. 1.

FIG. 12 is a flow chart illustrating an eleventh example method for tracking and performing actions using health data. This method may be performed by the system of FIG. 1.

FIG. 13 is a flow chart illustrating a twelfth example method for tracking and performing actions using health data. This method may be performed by the system of FIG. 1.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The description that follows includes sample systems, apparatuses, methods, and computer program products that embody various elements of the present disclosure. However, it should be understood that the described disclosure may be practiced in a variety of forms in addition to those described herein.

The present disclosure relates to tracking and performing actions using health data. The system is operable to use digital representations of biometrics to control access to identity information for people stored in an identification system. The system uses the stored identity information to track, evaluate, and/or correlate current and/or previously monitored health information for the people to perform one or more of a variety of actions. Such actions may include determining whether or not to allow the person access, providing attestations about a person's health information, routing the person based on one or more evaluations of the health information, and so on.

In this way, the identity system may be used to track and provide health data access and evaluations to a variety of different systems, enabling much wider tracking and evaluation than are possible with testing by individual different systems. This allows performance of functions related to health data tracking and evaluation that were previously not performable and enables health data tracking and evaluation in a less burdensome fashion and more efficiently while expending less work, eliminating unnecessary hardware and/or other components, and more efficiently using hardware, software, network, and/or other resources. This may improve the operation of systems involved by reducing unnecessary components, increasing the speed at which the systems perform operations, and/or reducing consumption of hardware, software, network, and/or other resources.

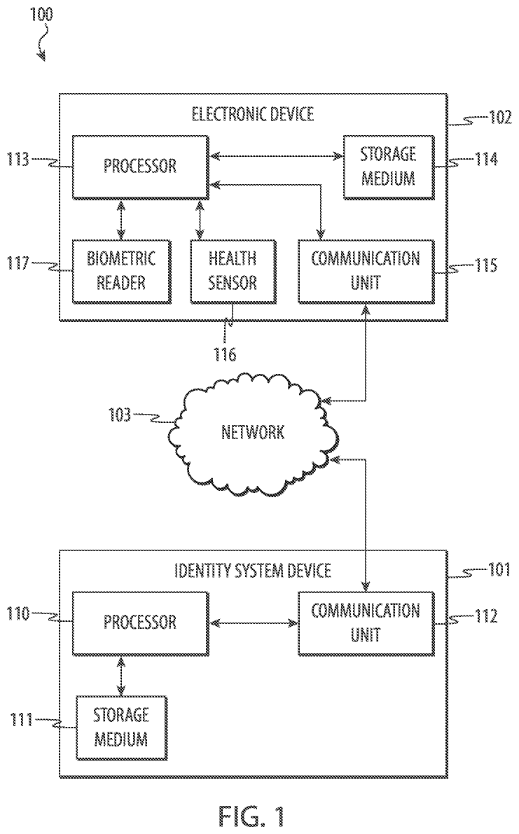

These and other embodiments are discussed below with reference to FIGS. 1-13. However, those skilled in the art will readily appreciate that the detailed description given herein with respect to these Figures is for explanatory purposes only and should not be construed as limiting.

FIG. 1 depicts a first example system 100 for tracking and performing actions using health data. The system 100 may include one or more identity system devices 101 that may be operative to communicate with one or more electronic devices 102 via one or more communication networks 103.

The identity system device 101 may store identity information (such as one or more names, addresses, telephone numbers, social security numbers, patient identification numbers or other identifiers, insurance data, financial data, health information (such as one or more temperatures, pupil dilation, medical diagnoses, immunocompromised conditions, medical histories, medical records, infection statuses, vaccinations, immunology data, results of antibody tests evidencing that a person has had a particular communicable illness and recovered, blood test results, saliva test results, and/or the like), and so on) associated with the identities of people (which may be verified identities, where the identities are verified as corresponding to the particular person named and/or where the identity information is verified as valid). Alternatively and/or additionally, some or all of the health information may be stored separately from the identity information but otherwise associated with the identity information (such as via one or more identifiers for such health information that may be stored in and/or otherwise associated with the identity information), such as in a Health Insurance Portability and Accountability Act (“HIPAA”) compliant or other data store or enclave and/or a blockchain and/or other auditable record or ledger. Such a data store or enclave may be stored on one or more different storage media than the identity information, or may be stored on the same storage medium or media and logically isolated from the identity information. The health information may be simultaneously and/or substantially simultaneously accessible as the identity information, such as where the identity information includes a health information identifier or key that may be used to access the separately stored health information. The identity system device 101 may control access to the identity information and/or the health information using identification information that is associated with the identity information. The identification information may include biometric data (which may include one or more digital representations of one or more fingerprints, blood vessel scans, palm-vein scans, voiceprints, facial images, retina images, iris images, deoxyribonucleic acid sequences, heart rhythms, gaits, and so on), one or more logins and/or passwords, authorization tokens, social media and/or other accounts, and so on. In various implementations, the identity system device 101 may allow the person associated with an identity to control access to the identity information, the health information, and/or other information (such as payment account information, health information (such as medical records, HIPAA protected information in order to be compliant with various legal restrictions, and so on), contact information, and so on. The identity system device 101 may control access to such information according to input received from the person. The identity system device 101 may be operable to communicate with the electronic device 102 in order to handle requests to provide the identity information and/or the health information, update and/or otherwise add to the identity information and/or the health information, provide attestations regarding and/or related to the identity information and/or the health information (such as whether or not a person is of a particular age, whether or not a person has a particular license or insurance policy, whether or not a person has been monitored as having particular health information, whether or not a person has had a particular vaccination, whether or not an antibody test evidences that a person has had a particular communicable illness and recovered, whether or not a person has a particular ticket or authorization, whether or not a person has been monitored as having particular antibodies, whether or not a person has been assigned a particular medical diagnosis, and so on), evaluate health information stored in the identity information and/or otherwise associated with the identity information and/or other information stored in the identity information, perform transactions, allow or deny access, route one or more persons, and/or perform one or more other actions.

By way of a first example, the electronic device 102 may obtain one or more digital representations (which may be in the form of one or more hashes of an electronic representation of the biometric and/or other data structures) of one or more biometrics from a person. The electronic device 102 may provide the digital representation of the biometric to the identity system device 101. The identity system device 101 may receive the digital representation of the biometric, use the digital representation of the biometric to retrieve one or more sets of identity information and/or health information associated with the person, and provide the retrieved identity information to the electronic device 102.

By way of a second example, the electronic device 102 may obtain one or more digital representations of one or more biometrics and health information from a person. By way of illustration, a thermal sensor may be located under a fingerprint scanner such that a temperature may be obtained from a person while an image of a fingerprint is obtained from the person. Alternatively, an image sensor may be used to capture retinal, iris, and/or other facial images of a person as biometric data as well as thermal images of the person during and/or contemporaneous with the retinal, iris, and/or other facial image capture for the purpose of determining the person's temperature. The electronic device may provide the digital representation of the biometric to the identity system device 101, which may use the digital representation of the biometric to retrieve a ticketing and/or other access account identifier from identity information associated with the person, communicate with a ticketing or other access system device using the ticketing and/or other access account identifier, receive information from the ticketing or other access system device regarding whether or not the person has a currently valid ticket and/or other access permissions (such as whether or not a person has a boarding pass for a flight, whether or not a person has a ticket for an event venue, whether or not a person is authorized to access a home or business, and so on), and provide the information from the ticketing or other access system device to the electronic device 102. The electronic device 102 may evaluate the information from the ticketing or other access system device to determine whether to grant or deny the person access. As part of determining whether or not to grant or deny the person access, the electronic device 102 may also evaluate the health information. For example, the electronic device 102 may compare the health information against allowed or denied health characteristics, such as where people with a temperature of 100 degrees Fahrenheit or above are prohibited access.

In a third example, the electronic device 102 may obtain one or more digital representations of one or more biometrics from a person. The electronic device may provide the digital representation of the biometric to the identity system device 101, which may use the digital representation of the biometric to retrieve health information from and/or otherwise associated with identity information associated with the person, evaluate the health information, and provide one or more results of the evaluation to the electronic device 102. For example, the identity system device 101 may notify the electronic device 102 whether or not the person has been recently monitored as having a fever, whether or not the person has been monitored as having particular antibodies, whether or not the person has received particular vaccinations, whether or not an antibody test evidences that the person has had a particular communicable illness and recovered, whether or not the person is immunocompromised, whether or not the person has been monitored as having contact with another person who tested positive for a bacterial or viral infection, and so on. The electronic device 102 may use such a notification as part of determining whether or not to allow the person access, whether or not to perform a transaction for the person, how to route the person, and/or to perform various other actions.

By way of a fourth example, the electronic device 102 may obtain one or more digital representations of one or more biometrics and health information from a person. The electronic device may provide the digital representation of the biometric and the health information to the identity system device 101, which may use the digital representation of the biometric to perform an action (such as determining whether or not to grant the person access, to perform an identification of the person, and so on). The identity system device 101 may store the health information for the person (and/or other information, such as information about the electronic device 102, the action performed, time and/or location data, and so on) in and/or otherwise associated with stored identity information for the person.

By way of a fifth example, the electronic device 102 may obtain one or more digital representations of one or more biometrics from a medical service provider who provides medical services to a person. The electronic device 102 may also obtain one or more medical records related to the medical services provided to the person as well as one or more digital representations of one or more biometrics from the person that were collected when the medical services provider provided the medical services to the person. The electronic device 102 may provide the identity system device 101 the one or more digital representations of the one or more biometrics from the medical service provider, the one or more medical records related to the medical services provided to the person, and/or the one or more digital representations of one or more biometrics from the person. The identity system device 101 may use such information to determine the corresponding identity information and/or health information to update with the one or more medical records related to the medical services provided to the person and/or other information, determine whether the update is authorized, determine whether or not the medical services provider is authorized to provide and/or update the identity information, determine whether or not the medical services provider is authorized to perform the medical services, and so on. The identity system device 101 may subsequently respond to authorized queries or perform other actions regarding the one or more medical records related to the medical services provided to the person and/or other information stored in and/or otherwise associated with the identity information.

In various implementations, the identity system device 101 may enable people associated with the identity information and/or the health information to opt in to storage, evaluation, and/or sharing of the identity information and/or the health information prior to any storage, evaluation, and/or sharing of such information. The identity system device 101 may enable the people to specifically opt in to some storage, evaluation, and/or sharing of such information without opting in to other storage, evaluation, and/or sharing of such information. Various configurations are possible and contemplated without departing from the scope of the present disclosure.

The techniques herein associating identity information with immunology and/or other health information may be used in healthcare contexts and/or in non-health care contexts. For example, the system 100 may be used to securely verify identity and provide verified health information to a medical service provider for a new patient onboarding remotely. By way of another example, the system 100 may be used to control access of people who many have communicable illnesses to areas that large groups of people congregate, such as cruise ships, sports stadiums, concert venues, churches, courthouses, schools, parks, restaurants, stores, and so on.

The identity system device 101 may be any kind of electronic device and/or cloud and/or other computing arrangement. Examples of such devices include, but are not limited to, one or more desktop computing devices, laptop computing devices, mobile computing devices, wearable devices, tablet computing devices, mobile telephones, smart phones, printers, displays, vehicles, kitchen appliances, entertainment system devices, digital media players, and so on. The identity system device 101 may include one or more processors 110 and/or other processing units or controllers, communication units 112, non-transitory storage media 111, and/or other components. The processor 110 may execute one or more sets of instructions stored in the non-transitory storage media 111 to perform various functions, such as receiving and/or storing biometric data and/or other identification information, receiving and/or storing identity information and/or health information, matching one or more received digital representations of biometrics and/or other identification information to stored data, retrieving identity information and/or health information associated with stored data matching one or more received digital representations of biometrics and/or other identification information, providing retrieved identity information and/or health information, communicating with the electronic device 102 via the network 103 using the communication unit 112, and so on.

Similarly, the electronic device 102 may be any kind of device. The electronic device 102 may include one or more processors 113 and/or other processing units and/or controllers, one or more non-transitory storage media 114 (which may take the form of, but is not limited to, a magnetic storage medium; optical storage medium; magneto-optical storage medium; read only memory; random access memory; erasable programmable memory; flash memory; and so on); one or more communication units 115; one or more health sensors 116 (such as a thermometer and/or other thermal sensor, a blood pressure sensor, a blood test sensor, a blood vessel scanner, a palm-vein scanner, a still image and/or video camera, a 2D and/or 3D image sensor, a saliva sensor, a breath sensor, a deoxyribonucleic acid sensor, a heart rhythm monitor, a microphone, sweat sensors, and so on); one or more biometric readers 117 (such as a fingerprint scanner, a blood vessel scanner, a palm-vein scanner, an optical fingerprint scanner, a phosphorescent fingerprint scanner, a still image and/or video camera, a 2D and/or 3D image sensor, a capacitive sensor, a saliva sensor, a deoxyribonucleic acid sensor, a heart rhythm monitor, a microphone, and so on), and/or one or more other components. The processor 113 may execute one or more sets of instructions stored in the non-transitory storage media 114 to perform various functions, such as using the biometric reader 117 to obtain one or more digital representations of one or more biometrics (such as a digital representation of a fingerprint, a blood vessel scan, a palm-vein scan, a voiceprint, a facial image, a retina image, an iris image, a deoxyribonucleic acid sequence, a heart rhythm, a gait, and so on) for a person, obtain health information for a person using the health sensor 116, communicate with the identity system device 101 via the network 103 using the communication unit 115, and so on.

Although the system 100 is illustrated and described as including particular components arranged in a particular configuration that perform particular functions, it is understood that this is an example. In various implementations, various arrangements of various components that perform various functions may be implemented without departing from the scope of the present disclosure.

For example, the biometric reader 117 and the health sensor 116 are illustrated and described as separate components. However, it is understood that this is an example. In some implementations, the biometric reader 117 may be operable to obtain health information for a person (such as an image sensor that is operable to obtain a retina or other image as biometric data that may also be evaluated to detect flushed skin, red or watery eyes, and/or other characteristics of influenza or other medical conditions). In such implementations and/or other implementations, the health sensor 116 may be omitted. In other implementations, the health sensor 116 may be operable to obtain biometric data from a person (such as a saliva sensor that is operable to obtain a saliva sample to evaluate whether or not particular antibodies are present that is also operable for use in deoxyribonucleic acid testing as biometric data). In such implementations and/or other implementations, the biometric reader 117 may be omitted. Various configurations are possible and contemplated without departing from the scope of the present disclosure.

By way of another example, the system 100 is illustrated and described as including one or more identity system devices 101 that communicate with one or more electronic devices 102. However, in other implementations, the identity system device 101 and/or the electronic device 102 may communicate with one or more other computing devices and/or systems, such as one or more age verification systems, payment processing systems, airline systems, ticketing and/or other access account systems, frequent flyer databases, watch lists, governmental databases, medical record databases, flight information databases, medical service provider systems, health information data stores or enclaves, and so on. Various configurations are possible and contemplated without departing from the scope of the present disclosure.

By way of yet another example, the system 100 is illustrated and described as storing health information in identity information associated with an identity. However, it is understood that this is an example. In some implementations, some or all of the health information may be stored separately from the identity information but otherwise associated with the identity information, such as in a HIPAA compliant or other data store or enclave. Such a data store or enclave may be stored on one or more different storage media than the identity information, or may be stored on the same storage medium or media and logically isolated from the identity information. The health information may be simultaneously and/or substantially simultaneously accessible as the identity information, such as where the identity information includes a health information identifier or key that may be used to access the separately stored health information. In various implementations, a health information account identifier may be stored in the identity information and used to access health information stored by a health information account system. Various configurations are possible and contemplated without departing from the scope of the present disclosure.

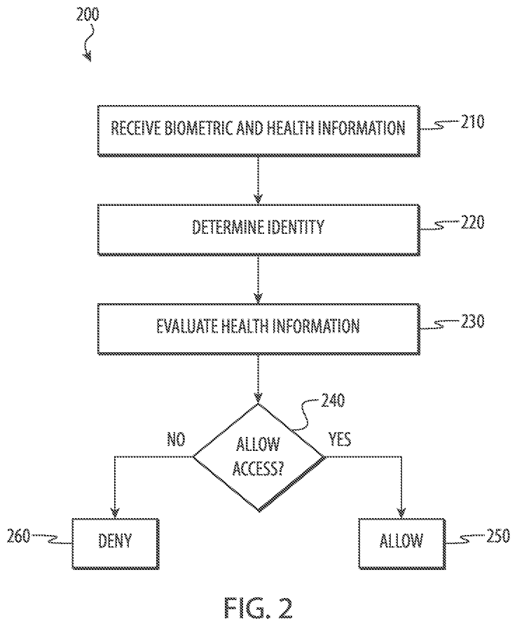

FIG. 2 is a flow chart illustrating a first example method 200 for tracking and performing actions using health data. This method 200 may be performed by the system 100 of FIG. 1.

At operation 210, an electronic device, such as the identity system device 101 of FIG. 1, may receive one or more digital representations of one or more biometrics and/or health information for one or more people. For example, the digital representation of the biometric may be in the form of one or more hashes of an electronic representation of the biometric and/or other data structures and/or may include one or more digital representations of one or more fingerprints, blood vessel scans, palm-vein scans, voiceprints, facial images, retina images, iris images, deoxyribonucleic acid sequences, heart rhythms, gaits, and so on. The digital representation of the biometric may be obtained via one or more biometric readers, such as a fingerprint scanner, a blood vessel scanner, a palm-vein scanner, an optical fingerprint scanner, a phosphorescent fingerprint scanner, a still image and/or video camera, a 2D and/or 3D image sensor, a capacitive sensor, a saliva sensor, a deoxyribonucleic acid sensor, a heart rhythm monitor, a microphone, and so on. The health information may include one or more temperatures, medical diagnoses, immunocompromised conditions, medical histories, medical records, infection statuses, vaccinations, immunology data, results of antibody tests evidencing that a person has had a particular communicable illness and recovered, blood test results, saliva test results, and so on. The health information may be obtained via one or more health sensors, such as a thermometer and/or other thermal sensor, a blood pressure sensor, a blood test sensor, a blood vessel scanner, a palm-vein scanner, a still image and/or video camera, a 2D and/or 3D image sensor, a saliva sensor, breath sensor, a deoxyribonucleic acid sensor, a heart rhythm monitor, a microphone, sweat sensors, and so on); one or more biometric readers (such as a fingerprint scanner, a blood vessel scanner, a palm-vein scanner, an optical fingerprint scanner, a phosphorescent fingerprint scanner, a still image and/or video camera, a 2D and/or 3D image sensor, a capacitive sensor, a saliva sensor, a deoxyribonucleic acid sensor, a heart rhythm monitor, a microphone, and so on). Alternatively and/or additionally, the health information may be received from one or more medical service provider systems, medical records storage systems, and so on.

At operation 220, the electronic device may use the digital representation of the biometric to determine an identity of the person. For example, the electronic device may compare the digital representation of the biometric to stored biometric data associated with identity information. Alternatively, the electronic device may transmit the digital representation of the biometric to an identity system, which may return identity information associated with the digital representation of the biometric.

At operation 230, the electronic device may evaluate the health information. For example, the electronic device may evaluate the health information to estimate whether or not there is a significant chance that the person has a communicable illness, such as a virus. By way of illustration, the electronic device may determine whether the health information indicates that the person has recent monitoring information indicating that the person has a fever, that the person has a high white blood cell count, and so on.

At operation 240, the electronic device may determine whether or not to allow the person access, such as access to pass through a security gate into a secure area. The electronic device may determine such based on the evaluation of the health information, the identity information (such as according to access permissions, ticket information, and so on indicated in and/or accessible via the identity information). If so, the flow may proceed to operation 250 where the electronic device allows access. Otherwise, the flow may proceed to operation 260 where the electronic device prohibits access.

By way of example, the electronic device may be a station at airport security. The station may include components that obtain a digital representation of a biometric for a person and a temperature for the person. The station may use the digital representation of the biometric to determine whether or not the person has a currently valid boarding pass for a flight at that airport and that the temperature does not indicate that the person might have a communicable disease. If so, the station may allow the person access to a secured area of the airport where a gate associated with the person's flight is located. Otherwise, the person may be denied access.

In another example, the electronic device may be an access gate at an event venue. The access gate may include components that obtain a digital representation of a biometric for a person and a thermal image of the person. The access gate may use the digital representation of the biometric to determine whether or not the person has a currently valid ticket to enter the event venue and that the thermal image does not indicate that the person might have a communicable disease. If so, the access gate may allow the person access to the event venue. Otherwise, the person may be denied access.

In still another example, an access control device at a restaurant may determine to deny access to a person because the person tests positive for a particular communicable illness. Identity information for the person may indicate that the person boarded a flight that morning and tested negative at security for the particular communicable illness. As a result, it may be determined that the person is likely to have contracted the particular communicable illness on the flight. Flight data of other passengers may be analyzed to determine other people who may be at risk in order to notify those people, add to their stored health information, track the spread of the particular communicable illness, and so on. In many situations, correlation of identity information stored for different people in such a way may be configured in order to comply with privacy regulations, such as HIPAA. For example, people may opt in to allow such data to be used in such a way, people may be able to specify how their information is accessed and/or used, health information may be anonymized to comply with privacy requirements and/or people's specifications, and so on.

In yet another example, the electronic device may be a station at a gym and/or other fitness and/or training facility. The station may include components that obtain a digital representation of a biometric for a person. The station may use the digital representation of the biometric to access health information for the person and determine whether or not the health information indicates that the person might have a communicable disease. If not, the station may allow the person access to the gym and/or other fitness and/or training facility. Otherwise, the person may be denied access. In some implementations, the station may determine a confidence level of the determination and may allow different levels of access for different determined confidence levels. By way of illustration, a person with a low confidence level may be allowed access conditional to use of protective gear (such as a mask) and temperature or other health information verification, a person with a middle confidence level may be allowed access conditional to use of protective gear without a temperature or other health information verification, and a person with a high confidence level may be allowed access without use of protective gear and/or temperature or other health information verification. Various configurations are possible and contemplated without departing from the scope of the present disclosure.

In some examples, a trustworthiness score may be determined for a person and/or used in determining whether or not to allow the person access and/or as part of making other determinations. Such a trust score may be based on publicly available financial and/or other information that indicates a general trustworthiness of the person, behavior patterns that tend to indicate a general trustworthiness of the person, watch lists, criminal behavior and/or civil wrongdoing, and so on. Such a trustworthiness score may also be based on other information, such as whether or not a person has ever provided false or misleading health information, whether or not a person has ever withheld information regarding a health risk, whether or not a person has ever asserted health and later been found to be ill (such as receiving medical services to treat a communicable illness shortly after asserting that they had not been exposed to the communicable illness and so on), whether or not the person or a connected person is diagnosed with a communicable illness after voluntarily attending a situation where people with a risk of the communicable illness were not supposed to attend, and so on. Such a trustworthiness score may be used to allow or deny access, to supplement other procedures (such as where people with high trustworthiness scores only need to provide a non-elevated temperature for access but people with low trustworthiness scores need to provide the temperature and acceptable results of a vaccination and/or antibody test), and so on. Various configurations are possible and contemplated without departing from the scope of the present disclosure.

In various examples, this example method 200 may be implemented as a group of interrelated software modules or components that perform various functions discussed herein. These software modules or components may be executed within a cloud network and/or by one or more computing devices, such as the identity system device 101 and/or the electronic device 102 of FIG. 1.

Although the example method 200 is illustrated and described as including particular operations performed in a particular order, it is understood that this is an example. In various implementations, various orders of the same, similar, and/or different operations may be performed without departing from the scope of the present disclosure.

For example, operation 210 is illustrated and described as receiving the health information. However, it is understood that this is an example. In various implementations, the electronic device may instead access health information stored in the identity information. Various configurations are possible and contemplated without departing from the scope of the present disclosure.

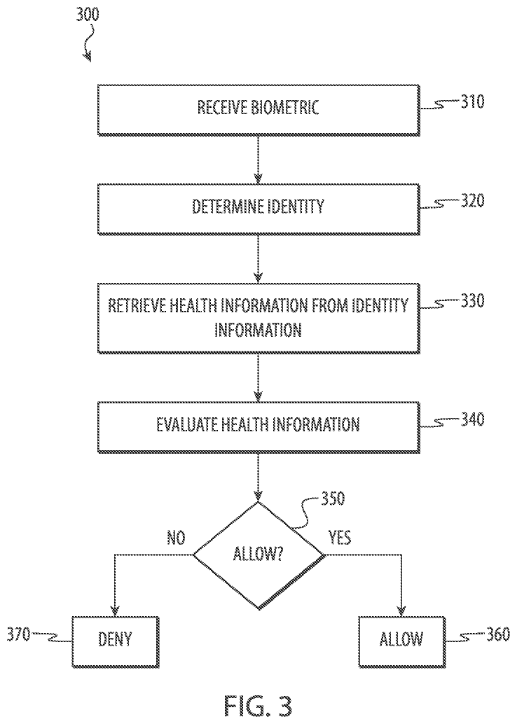

FIG. 3 is a flow chart illustrating a second example method 300 for tracking and performing actions using health data. This method 300 may be performed by the system 100 of FIG. 1.

At operation 310, an electronic device, such as the identity system device 101 of FIG. 1, may receive a digital representation of a biometric for a person. At operation 320, the electronic device may use the digital representation of the biometric to determine an identity of the person. At operation 330, the electronic device may retrieve health information from identity information stored associated with the identity and/or a data store or enclave that is associated with the identity information. The health information may have been stored as part of previous identifications, access authorizations, and/or other actions performed by the electronic device or a related device. Alternatively, the health information may have been provided to store in and/or otherwise be associated with the identity information from one or more medical provider systems, medical records storage systems, and so on.

At operation 340, the electronic device may evaluate the health information. The electronic device may determine whether or not the health information meets minimum health information requirements, such as that the person's recently monitored temperature is 99 degrees Fahrenheit or below, the person has been vaccinated for a communicable illness (such as measles, mumps, and so on), results of an antibody test evidences that a person has had a particular communicable illness and recovered, the person has no record of travel to an area suffering from an epidemic or exposure to an infected person, and so on. At operation 350, the electronic device may determine whether or not to allow the person access. If so, the flow may proceed to operation 360 where the electronic device allows access. Otherwise, the flow may proceed to operation 370 where the electronic device denies access.

For example, an entry control mechanism at the door of a restaurant may obtain a digital representation of a biometric for a person and use such to access identity information and/or health information stored for the person indicating that the person was recently monitored to have a 101 degree Fahrenheit fever. As such, the entry control mechanism may deny the person access.

By way of another example, an airport security station (and/or a security station at a location other than an airport, such as a gym and/or other fitness and/or training facility) may obtain a digital representation of a biometric for a person and use such to determine that the person recently was on a flight or in an area of an event venue with another person who tested positive for measles. As such, the airport security station may deny the person access until the identity information and/or the health information for the person is updated to indicate that the person tests negative for measles, is vaccinated for measles, an antibody test evidences that the person has had a particular communicable illness and recovered, and so on. In some implementations, the system storing the identity information and/or the health information may correlate identity information stored for different people to identify who may have been exposed to the person with measles and notify such persons (such as via electronic devices or communication information stored in the identity information) to provide testing and/or vaccination data and/or antibody test results indicating that the person is not likely to transmit measles to other people before the system will be usable to grant them one or more kinds of access. Various configurations are possible and contemplated without departing from the scope of the present disclosure.

In various examples, this example method 300 may be implemented as a group of interrelated software modules or components that perform various functions discussed herein. These software modules or components may be executed within a cloud network and/or by one or more computing devices, such as the identity system device 101 and/or the electronic device 102 of FIG. 1.

Although the example method 300 is illustrated and described as including particular operations performed in a particular order, it is understood that this is an example. In various implementations, various orders of the same, similar, and/or different operations may be performed without departing from the scope of the present disclosure.

For example, operation 340 is illustrated and described as evaluating the health information. However, it is understood that this is an example. In other implementations, an indication regarding whether or not the person should be allowed and/or denied access based on a health risk may be retrieved instead of the health information. Various configurations are possible and contemplated without departing from the scope of the present disclosure.

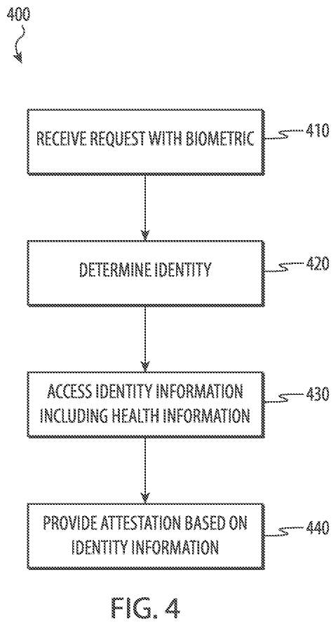

FIG. 4 is a flow chart illustrating a third example method 400 for tracking and performing actions using health data. This method 400 may be performed by the system 100 of FIG. 1.

At operation 410, an electronic device, such as the identity system device 101 of FIG. 1, may receive a request with a digital representation of a biometric for a person. The request may be a request for a particular attestation, such as that the person is authorized to access an area (such as that the person has a valid boarding pass for a relevant flight, the person has a valid ticket for a relevant event at an event venue, the person has an appointment for medical services, and so on), an attestation that the person is not a health risk, an attestation that the person has been vaccinated for a particular illness, an attestation that results of an antibody test evidences that a person has had a particular communicable illness and recovered, an attestation that the person has recently been monitored with a normal temperature, and so on. Additionally or alternatively, the request may be a request for a transaction (such as to process payment for a transaction, to place an order, and so on).

At operation 420, the electronic device may use the digital representation of the biometric to determine an identity for the person. At operation 430, the electronic device may access identity information (which includes and/or is associated with health information) that is associated with the identity. At operation 440, the electronic device may provide one or more attestations based on the identity information, such as based on the included and/or associated health information. By way of illustration, the electronic device may provide one or more attestations that the person has a valid boarding pass for a relevant flight, the person has a valid ticket for a relevant event at an event venue, the person has an appointment for medical services, the person is not a health risk, the person cannot be confirmed to not be a health risk, the person has been vaccinated for a particular illness, results of an antibody test evidence that the person has had a particular communicable illness and recovered, results of an antibody test do not evidence that the person has had a particular communicable illness and recovered, the person has not been vaccinated for a particular illness, the person has recently been monitored with a normal temperature, the person has not recently been monitored with a normal temperature, the person has recently been monitored with an abnormal temperature, and so on.

For example, a check-in station at a medical service provider office (and/or a station at a location other than a medical service provider office, such as a gym and/or other fitness and/or training facility) may receive a digital representation of a biometric for a person and use that information to provide one or more attestations that the person has an appointment for medical services and that the person has been vaccinated for a particular communicable illness or that results of an antibody test evidence that the person has had a particular communicable illness and recovered. As such, medical service provider personnel may allow the person to wait for the person's appointment in a waiting room. Alternatively, the person may not be allowed to wait if the check-in station provides an attestation that the person does not have an appointment for medical services, or may be allowed to wait in an isolated room if the check-in station provides an attestation that the person has not been vaccinated for the particular communicable illness or that results of an antibody test evidence that the person has had the particular communicable illness and recovered.

By way of another example, an entry mechanism at a nursing home may transmit a request to an identity system device for an attention that a person associated with a digital representation of a biometric is not a known health risk. The identity system device may use the digital representation of the biometric to determine the identity for the person, access identity information that includes and/or is otherwise associated with health information for the person, and provide an attestation based on the health information and/or other information included in and/or otherwise associated with the identity information that the person is either not a health risk or cannot be determined to not be a health risk. The entry mechanism may allow the person access to the nursing home if the attestation indicates that the person is not a health risk, and may deny the person access to the nursing home if the attestation indicates that the person cannot be determined to not be a health risk.

In various examples, this example method 400 may be implemented as a group of interrelated software modules or components that perform various functions discussed herein. These software modules or components may be executed within a cloud network and/or by one or more computing devices, such as the identity system device 101 and/or the electronic device 102 of FIG. 1.

Although the example method 400 is illustrated and described as including particular operations performed in a particular order, it is understood that this is an example. In various implementations, various orders of the same, similar, and/or different operations may be performed without departing from the scope of the present disclosure.

For example, operation 440 is illustrated and described as providing the attestation based on the identity information and/or the health information. However, it is understood that this is an example. In some implementations, the electronic device may determine that there is insufficient information to make the attestation and may respond to the request by indicating such. Various configurations are possible and contemplated without departing from the scope of the present disclosure.

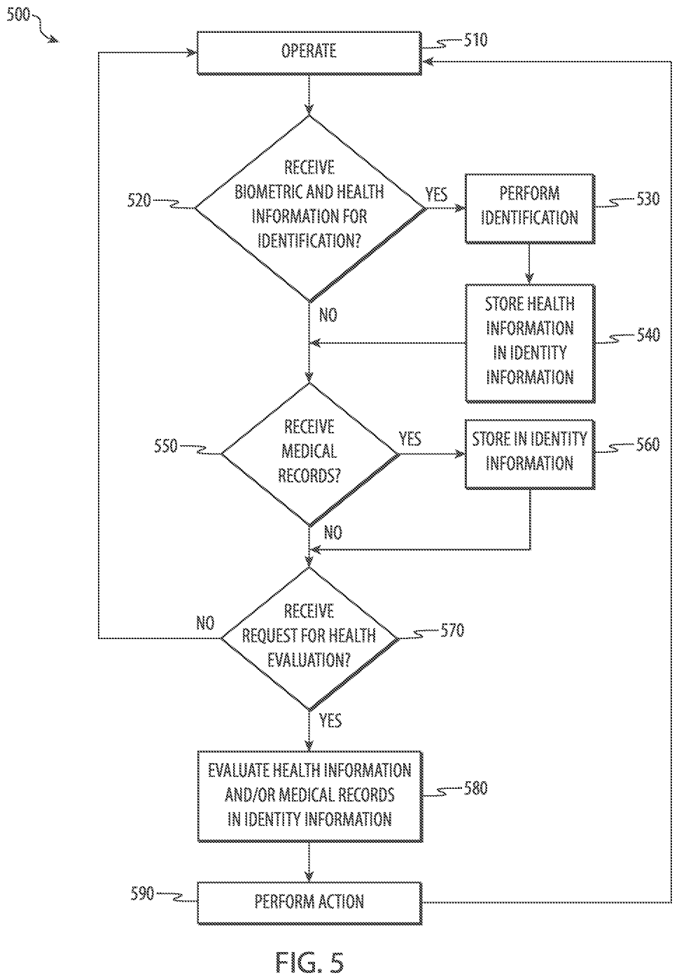

FIG. 5 is a flow chart illustrating a fourth example method 500 for tracking and performing actions using health data. This method 500 may be performed by the system 100 of FIG. 1.

At operation 510, an electronic device, such as the identity system device 101 of FIG. 1, may operate. At operation 520, the electronic device may determine whether or not a digital representation of a biometric and health information for a person is received for purposes of performing a biometric identification. If so, the flow may proceed to operation 530 where the electronic device may perform the biometric identification. Otherwise, the flow may proceed to operation 550.

After the electronic device performs biometric identification at operation 530, the flow may proceed to operation 540 where the electronic device may store the health information in and/or associated with identity information associated with an identity corresponding to the identification. The flow may then proceed to operation 550.

At operation 550, the electronic device may determine whether or not one or more medical records are received that are associated with an identity. If so, the flow may proceed to operation 560 where the received medical record or records are stored in and/or associated with identity information associated with the identity before the flow may proceed to operation 570. Otherwise, the flow may proceed directly to operation 570.

At operation 570, the electronic device may determine whether or not a request for a health evaluation is received. If not, the flow may return to operation 510 where the electronic device may continue to operate. Otherwise, the flow may proceed to operation 580 where the electronic device may evaluate health information and/or one or more medical records otherwise stored in and/or associated with identity information associated with an identity. The flow may then proceed to 590 where the electronic device may perform one or more actions based on the evaluation before the flow returns to operation 510 and the electronic device continues to operate.

For example, the action may include the electronic device providing the results of the evaluation, one or more attestations based on the results of the evaluation, an indication that the person is not a known health risk, an indication that the person cannot be confirmed to not be a known health risk, routing the person based on the results of the evaluation, an instruction to route the person based on the results of the evaluation, and so on. In some examples, the action may be specified in the request.

In various examples, this example method 500 may be implemented as a group of interrelated software modules or components that perform various functions discussed herein. These software modules or components may be executed within a cloud network and/or by one or more computing devices, such as the identity system device 101 and/or the electronic device 102 of FIG. 1.

Although the example method 500 is illustrated and described as including particular operations performed in a particular order, it is understood that this is an example. In various implementations, various orders of the same, similar, and/or different operations may be performed without departing from the scope of the present disclosure.

For example, operation 530 is illustrated and described as performing the biometric identification. However, it is understood that this is an example. In some implementations, the electronic device may not be able to determine an identity using the received digital representation of the biometric. In such an implementation, the electronic device may return an error and may not store the health information in and/or otherwise associated with identity information associated with an identity. Various configurations are possible and contemplated without departing from the scope of the present disclosure.

By way of another example, operation 560 is illustrated and described as storing one or more medical records in and/or otherwise associated with identity information associated with an identity. However, it is understood that this is an example. In some implementations, the electronic device may determine whether or not the one or more medical records are verified before storing, and/or that the storage is authorized. In still other implementations, the electronic device may be unable to determine an identity associated with the one or more medical records and thus may instead determine that an error occurred rather than storing the one or more medical records. Various configurations are possible and contemplated without departing from the scope of the present disclosure.

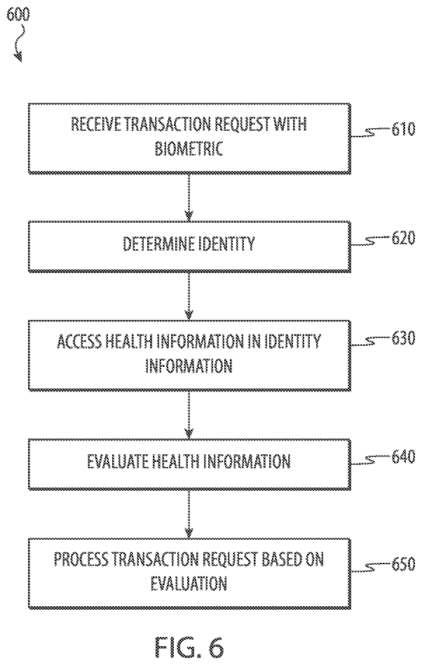

FIG. 6 is a flow chart illustrating a fifth example method 600 for tracking and performing actions using health data. This method 600 may be performed by the system 100 of FIG. 1.

At operation 610, an electronic device, such as the identity system device 101 of FIG. 1, may receive a request for a transaction with a digital representation of a biometric for a person. At operation 620, the electronic device may determine an identity using the digital representation of the biometric. At operation 630, the electronic device may access health information stored in and/or otherwise associated with identity information associated with the identity. At operation 640, the electronic device may evaluate the health information. At operation 650, the electronic device may process the transaction request based on the evaluation.

For example, the transaction request may be a request to book a ticket for a flight or entrance to an event venue (and/or another location, such as a gym and/or other fitness and/or training facility). The transaction request may specify that a particular vaccination and/or results of an antibody test evidencing that the person has had a particular communicable illness and recovered and no health characteristic indicative of a particular communicable illness is a requirement for the ticket. As such, the electronic device may process the transaction request to book the ticket if the evaluation indicates that the person has the particular vaccination and/or that results of an antibody test evidencing that the person has had a particular communicable illness and recovered and has no health characteristic indicative of the particular communicable illness and may process the transaction request to not book the ticket if the evaluation indicates that the person does not have the particular vaccination and/or results of an antibody test do not evidence that the person has had a particular communicable illness and recovered and/or has any health characteristic indicative of the particular communicable illness.

Alternatively, the transaction request may specify to book a first ticket if the evaluation indicates that the person has the particular vaccination and/or results of an antibody test evidencing that the person has had a particular communicable illness and recovered and has no health characteristic indicative of the particular communicable illness and to book a second ticket if the evaluation indicates that the person does not have the particular vaccination and/or results of an antibody test do not evidence that the person has had a particular communicable illness and recovered and/or has any health characteristic indicative of the particular communicable illness. For example, seating or other ticketing arrangements may be assigned to isolate vulnerable people from people who may be at risk of having the particular communicable illness, to place vaccinated and/or recovered and/or otherwise immune people between other people, and so on. Various configurations are possible and contemplated without departing from the scope of the present disclosure.

By way of another example, the transaction request may specify that purchase of masks or other medical protective gear is to be restricted to people who have contracted a communicable illness and/or immunocompromised people. As such, the transaction request may be processed to approve the transaction request if the evaluation indicates that the person has contracted a communicable illness and/or is immunocompromised and to reject the transaction if the evaluation indicates that the person has not contracted a communicable illness and is not immunocompromised. Various configurations are possible and contemplated without departing from the scope of the present disclosure.

In various examples, this example method 600 may be implemented as a group of interrelated software modules or components that perform various functions discussed herein. These software modules or components may be executed within a cloud network and/or by one or more computing devices, such as the identity system device 101 and/or the electronic device 102 of FIG. 1.

Although the example method 600 is illustrated and described as including particular operations performed in a particular order, it is understood that this is an example. In various implementations, various orders of the same, similar, and/or different operations may be performed without departing from the scope of the present disclosure.

For example, operations 640-650 are illustrated and described as evaluating the health information and processing the transaction request based on the evaluation. However, it is understood that this is an example. In some implementations, the operation of evaluating the health information may be omitted and the transaction request may instead be processed based on the health information as opposed to processing the transaction request based on any evaluation of the health information. Various configurations are possible and contemplated without departing from the scope of the present disclosure.

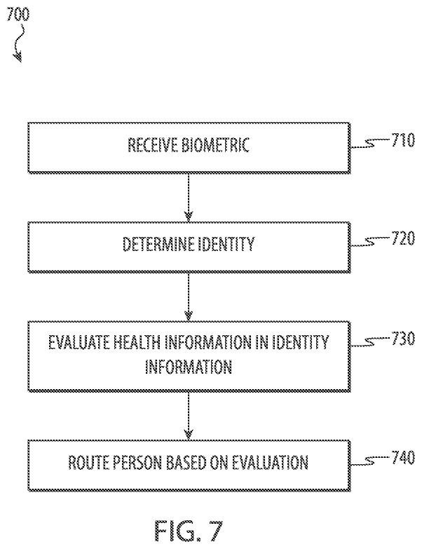

FIG. 7 is a flow chart illustrating a sixth example method 700 for tracking and performing actions using health data. This method 700 may be performed by the system 100 of FIG. 1.

At operation 710, an electronic device, such as the identity system device 101 of FIG. 1, may receive a digital representation of a biometric for a person. At operation 720, the electronic device may determine an identity using the digital representation of the biometric. At operation 730, the electronic device may evaluate health information stored in and/or otherwise associated with identity information associated with the identity. At operation 740, the electronic device may route the person based on the evaluation.

For example, people with tickets for a flight or entrance to an event venue may be assigned to a section without being assigned specific seats. An access control device upon arrival may obtain a digital representation of a biometric on arrival, determine identity using the digital representation of the biometric, evaluate health information stored in and/or otherwise associated with identity information associated with the identity, and route people to particular seats based on the evaluation. By way of illustration, seating or other ticketing arrangements may be assigned to isolate vulnerable people from people who may be at risk of having the particular communicable illness, to place vaccinated and/or recovered and/or otherwise immune people between other people, and so on. Various configurations are possible and contemplated without departing from the scope of the present disclosure.

By way of another example, a nursing home or other facility (such as a gym and/or other fitness and/or training facility) may have separate elevators for people who may have contracted a communicable illness and for immunocompromised people. As such, an access control mechanism for the two elevators may be used to determine people who may have contracted a communicable illness, determine immunocompromised people, and route the two sets of people to the corresponding elevators. Various configurations are possible and contemplated without departing from the scope of the present disclosure.

In various examples, this example method 700 may be implemented as a group of interrelated software modules or components that perform various functions discussed herein. These software modules or components may be executed within a cloud network and/or by one or more computing devices, such as the identity system device 101 and/or the electronic device 102 of FIG. 1.

Although the example method 700 is illustrated and described as including particular operations performed in a particular order, it is understood that this is an example. In various implementations, various orders of the same, similar, and/or different operations may be performed without departing from the scope of the present disclosure.

For example, the operations 730-740 are illustrated and described as evaluating the health information and routing the person based on the evaluation. However, it is understood that this is an example. In some implementations, the operation of evaluating the health information may be omitted and the person may instead be routed based on the health information as opposed to routing the person based on any evaluation of the health information. Various configurations are possible and contemplated without departing from the scope of the present disclosure.

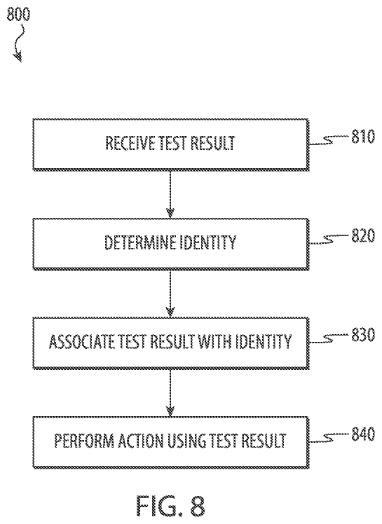

FIG. 8 is a flow chart illustrating a seventh example method 800 for tracking and performing actions using health data. This method 800 may be performed by the system 100 of FIG. 1.

At operation 810, an electronic device, such as the identity system device 101 of FIG. 1, may receive a test result for a person. For example the test result may be a rapid result and/or other blood test, saliva test, and/or other test that determines whether or not a person may have contracted a communicable illness. By way of another example, the test result may be an antibody test to determine whether or not the person has contracted a communicable illness and recovered.

At operation 820, the electronic device may determine an identity of the person. For example, the electronic device may obtain one or more digital representations of one or more biometrics for the person, compare the digital representation of the biometric to stored biometric data corresponding to verified identities, monitor that the person for whom the test result is received is the same person as who provided the digital representation of the biometric, and so on.

At operation 830, the electronic device may associate the test result with the identity. The electronic device may store the test result in identity information for the person, store the test result in health information for the person that is associated with the identity information, and so on.

At operation 840, the electronic device may perform one or more actions using the test result. For example, the electronic device may provide one or more attestations regarding the test result, may route the person based on the test result, allow and/or deny access based on the test result, and so on.

By way of example, a person may take a rapid result blood test at a kiosk at a drug store. The kiosk may include a fingerprint sensor that obtains an image of the person's fingerprint at the same time that the kiosk pricks the person's finger to obtain a blood sample to test, which may ensure that the blood is from the same person as the fingerprint image. The person may be identified using the fingerprint image and test results of the rapid result blood test may be associated with the identity of the person. Various configurations are possible and contemplated without departing from the scope of the present disclosure.

In another example, the person may take a test at a kiosk that may be video monitored. The person may be identified from the video using facial recognition techniques and test results (whether communicated by the kiosk, monitored on the video, and so on) may be associated accordingly. Various configurations are possible and contemplated without departing from the scope of the present disclosure.

By way of another example, a person may take a test at a kiosk and use an app for an identity system executing on a mobile device to monitor the test, provide one or more digital representations of biometrics in order to identify himself, and so on. Various configurations are possible and contemplated without departing from the scope of the present disclosure.

In another example, a person may obtain a home test for a communicable illness. The person may complete the home test and self-enter the test result into an app for an identity system executing on a mobile device. The app may obtain one or more digital representations of one or more biometrics from the person in order to determine an identity for the person and associate the test results with the identity.

In some examples, a trustworthiness score may be determined for a person and/or used in determining whether or not to allow the person access and/or as part of making other determinations. Such a trust score may be based on publicly available financial and/or other information that indicates a general trustworthiness of the person, behavior patterns that tend to indicate a general trustworthiness of the person, watch lists, criminal behavior and/or civil wrongdoing, and so on. Such a trustworthiness score may also be based on other information, such as whether or not a person has ever provided false or misleading health information, whether or not a person has ever withheld information regarding a health risk, whether or not a person has ever asserted health and later been found to be ill (such as receiving medical services to treat a communicable illness shortly after asserting that they had not been exposed to the communicable illness and so on), whether or not the person or a connected person is diagnosed with a communicable illness after voluntarily attending a situation where people with a risk of the communicable illness were not supposed to attend, and so on. Such a trustworthiness score may be used in a variety of different ways. For example, the test result self-enter example above may be restricted to people with trustworthiness scores above a threshold, whereas people with trustworthiness scores at or below the threshold may be required to validate the test results in some way. Various configurations are possible and contemplated without departing from the scope of the present disclosure.

For example, the app may be used to capture an image and/or other proof of the test result of the home test, which may include obtaining a time stamp and/or other test proof. By way of another example, the app may be used to capture video of the person taking the home test as well as the test result to ensure that the person who provides the digital representation of the biometric is the same person who took the test. Various configurations are possible and contemplated without departing from the scope of the present disclosure.

In another example, the home test may be registered to the identity of the person. By way of illustration, the app may be used to scan a Quick Read code or other bar code on the home test and/or serial number or other identifier on the test as well as one or more digital representations of one or more biometrics for the person. This may be used to associate the particular home test with the identity of the person, which may deter the person from having another person take the test and/or falsely reporting the test result. By way of another illustration, the home test may be associated with the identity before being provided to the person, such as where the person has been prompted to take the home test and is provided a home test already registered to his identity. In some examples, the home test may auto report results and thus the auto reported test results may be associated with the identity to whom the test is registered. In various examples, the app may be used to monitor the person taking the test to ensure that the identity to whom the home test is registered corresponds to the person who takes the test. Various configurations are possible and contemplated without departing from the scope of the present disclosure.

In some example implementations where trustworthiness scores may be determined, people who cannot be determined to be trustworthy may not be allowed to take home tests and may instead have to take monitored tests. In other example implementations, less trustworthy people may be required to submit to higher levels of verification for home tests, such as video monitoring during testing, whereas trustworthy people may be allowed to self-enter test results. The degree of certainty associated with the test result may be associated with the trustworthiness score of the person, the verifications associated with the home test, and so on. In some examples, the degree of certainty associated with the test result may be evaluated as part of evaluating the health information, such as allowing access if the degree of certainty is at least a threshold and denying otherwise, and so on. Various configurations are possible and contemplated without departing from the scope of the present disclosure.

In various examples, the test results may be digitally notarized by the person and/or another person. For example, the person and/or another person may provide one or more digital representations of one or more biometrics when test results are provided in order for the person and/or the other person to attest that the provided test results are accurate. In some situations, a trustworthy person may attest to the test results for a less trustworthy person so that the less trustworthy person may use the home test, may self-enter the test result, and so on. By way of illustration, the other person may be a pharmacist, a doctor, a nurse, and so on. Various configurations are possible and contemplated without departing from the scope of the present disclosure.

Although the above describes a home test, it is understood that this is an example. In other examples, the above techniques may be used for a variety of administered tests and/or other kinds of tests without departing from the scope of the present disclosure. Various configurations are possible and contemplated without departing from the scope of the present disclosure.

In some implementations, multiple test results may be obtained and/or associated with a person's identity. For example, some antibody tests may have a high degree of false negative results (such as 20%, 30%, or even higher) such that the antibody tests may need to be repeated multiple times to verify that a person has previously overcome a particular communicable illness. As such, multiple test results may be obtained and associated with the identity and a later positive test result indicating that the person has previously overcome a particular communicable illness may override a previous negative test result that falsely indicated that the person had not previously overcome the particular communicable illness. By way of illustration, the person may know and/or suspect that he has already had and overcome the particular communicable illness. The person may obtain and take a home test, which may not prove that the person has overcome the particular illness. The person may then obtain another home test and retake until proof is obtained. Various configurations are possible and contemplated without departing from the scope of the present disclosure.

In other implementations, a person providing multiple test results may indicate that the person is trying to obtain a falsely positive determination that the person desires instead of ameliorating a falsely negative determination. For example, instead of an antibody test that never indicates that a person has overcome an illness that the person has not but sometimes indicates that the person has not overcome an illness that the person has, the reverse may be true. As such, the person may repeat the test hoping to obtain a clearance that the person does not deserve. In such a case, multiple test results may be tracked to determine that the person is trying to game the tests and override a clearance that might otherwise be provided. In some implementations of such examples, a trustworthiness score of the person may be evaluated to determine when a person may be attempting to game the tests and/or when the person may be appropriately pursuing multiple tests for certainty. Various configurations are possible and contemplated without departing from the scope of the present disclosure.

In various examples, this example method 800 may be implemented as a group of interrelated software modules or components that perform various functions discussed herein. These software modules or components may be executed within a cloud network and/or by one or more computing devices, such as the identity system device 101 and/or the electronic device 102 of FIG. 1.

Although the example method 800 is illustrated and described as including particular operations performed in a particular order, it is understood that this is an example. In various implementations, various orders of the same, similar, and/or different operations may be performed without departing from the scope of the present disclosure.

For example, the method 800 is illustrated and described in the context of receiving a test result. However, it is understood that this is an example. In various implementations, a health measurement and/or other data regarding the person may be obtained instead of a test result. By way of illustration, a temperature reading may be obtained for the person instead of a formal test result. Various configurations are possible and contemplated without departing from the scope of the present disclosure.

By way of another example, the method 800 is illustrated and described as associating the test result with the identity. However, it is understood that this is an example. In some implementations, the test result may be reported and/or verified as being a test result for the identity without storage and/or formal association with the identity. Various configurations are possible and contemplated without departing from the scope of the present disclosure.

Test results (such as antibody test results) and/or other health information may be associated with identity information for a person in a variety of different ways. For example, a person may indicate that a test result is his and request association. By way of another example, an entity associated with the test results (such as a testing facility, a provider of a home test kit, a pharmacy, and so on) may communicate the test results and the test results may then be associated with identity information for a person. Regardless how requests for association of such test results and/or other health information may be initiated, one or more electronic devices receiving such a request may verify correspondence between the test results and one or more identities before associating the test results with one or more sets of identity information. This verification may be performed in a variety of different ways.

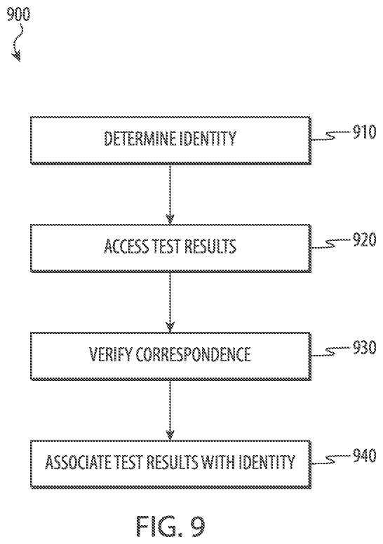

FIG. 9 is a flow chart illustrating an eighth example method 900 for tracking and performing actions using health data. This method 900 may be performed by the system 100 of FIG. 1.

At operation 910, an electronic device, such as the identity system device 101 of FIG. 1, may determine an identity of a person. For example, the electronic device may compare a digital representation of a biometric for the person with stored biometric data associated with identity information in order to determine the identity of the person. At operation 920, the electronic device may access test results. The electronic device may access the test results in a variety of different ways. In some implementations, the electronic device may receive the test results from an entity associated with the test results (such as a testing facility, a provider of a home test kit, a pharmacy, and so on). In other implementations, the electronic device may receive information from the person that the electronic device may use to look up and/or otherwise access the test results (such as a test identifier and so on). In still other implementations, the electronic device may receive the test results from the person. In such an implementation, the electronic device may then communicate an entity associated with the test results (such as a testing facility, a provider of a home test kit, a pharmacy, and so on) to verify that the received test results are valid. In some examples, the test results may be machine readable, encrypted, and/or otherwise stored in such a way that the test results are not readable by the person and must be accessed via the electronic device or other authorized device in order for the test results to be comprehensible. This may prevent the person from learning of adverse test results and then not associating those adverse test results with his identity information, particularly when the test results are obtained from a home test as opposed to a monitored test, which a person may be more able to avoid reporting if the person knew of an adverse result before reporting of the result.

At operation 930, the electronic device may verify correspondence between the test results and the identity. In some implementations, this may involve comparing biographic and/or other information stored in and/or otherwise associated with identity information for the person with biographic and/or other information associated with the test results. For example, such information may include first name, middle name or initial, last name, insurance information, address, gender, social security number and/or other identifier, and so on. When the electronic device determines that the biographic and/or other information stored in and/or otherwise associated with identity information for the person corresponds to that from the test results, the electronic device may determine that the test results are for the person and determine that the correspondence between the test results and the identity is verified. In some examples, the electronic device may determine that that one or more items of the biographic and/or other information stored in and/or otherwise associated with identity information for the person matches that from the test results in order to verify correspondence between the test results and the identity. In other examples, the electronic device may determine that a threshold certainty level is met based on similarity between one or more items of the biographic and/or other information stored in and/or otherwise associated with identity information for the person and that from the test results in order to verify correspondence between the test results and the identity (such as where one lists a full middle name and the other lists a middle initial, where one lists a current address and the other lists an old address, and so on). In other implementations, the electronic device may verify correspondence by verifying that the test results are not associated with health information that is inconsistent with health information stored in and/or otherwise associated with the identity information. For example, the test results may include data regarding age of the test subject, blood type of the test subject, DNA of the test subject, gender of the test subject, and/or other health information about the test subject that may have been obtained from a sample related to the test (such as a blood sample, a DNA sample, a mucus sample, and so on) and such data may be compared to health information stored in and/or otherwise associated with the identity information. By way of illustration, the electronic device may verify correspondence by ensuring that the test results are not associated with an age inconsistent with that of the person, blood type inconsistent with that of the person, DNA inconsistent with that of the person, gender inconsistent with that of the person, and/or other health information inconsistent with that of the person. Such data may be gathered during testing. In implementations where the test is a home test kit, such data may be gathered by collecting test kits after test completion and later testing samples included in the collected test kits in order to obtain the data, whether routinely and/or in situations where additional verification is determined to be performed. In still other implementations, identities of people may be biometrically determined upon entry to a testing facility and tests results may be verified as corresponding to those identities based upon the identities biometrically determined upon entry. Various configurations are possible and contemplated without departing from the scope of the present disclosure.

At operation 940, after verifying the correspondence, the electronic device may associate the test results with the identity. This may include storing the test results in the identity information, storing the test results in a data store or enclave and/or a blockchain and/or other auditable record or ledger associated with the identity, and so on. For example, a blockchain and/or other auditable record or ledger may include one or more data blocks with one or more test result identifiers and/or one or more identifiers for and/or associated with the identity. The one or more test result identifiers and/or one or more identifiers for and/or associated with the identity may be used to associate the test results with identity information for the person, such as where the one or more test result identifiers and/or one or more identifiers for and/or associated with the identity are stored in the identity information and used to access the test results from the blockchain and/or other auditable record or ledger.

In various examples, this example method 900 may be implemented as a group of interrelated software modules or components that perform various functions discussed herein. These software modules or components may be executed within a cloud network and/or by one or more computing devices, such as the identity system device 101 and/or the electronic device 102 of FIG. 1.

Although the example method 900 is illustrated and described as including particular operations performed in a particular order, it is understood that this is an example. In various implementations, various orders of the same, similar, and/or different operations may be performed without departing from the scope of the present disclosure.

For example, the method 900 illustrates and describes verifying correspondence between accessed test results and a determined identity. However, it is understood that this is an example. In other implementations, correspondence between a test and an identity may instead be verified, whether prior to, subsequent to, and/or during determination of the test results. For example, a home test kit may be registered to an identity when purchased and correspondence between the home test kit and the identity may be verified. An identity (and/or family, household, and/or other relationship associated with an identity) may be only able to be associated with the purchase of one home test kit in order to prevent and/or reduce the possibility that a person will obtain multiple test kits and report that person's results for multiple different people (and/or otherwise reduce the incentive for the person to attempt to invalidly associate a test result with his and/or another person's identity). Exceptions may be made for situations where a test has a high false negative rate, such as where a test with a high false negative rate may be overridden by a later test with sufficient verification that both tests were taken by the same person, where a less accurate test may be overridden by a later and more accurate test, where a home test may be overridden by a monitored test, where multiple tests are taken into account when determining a confidence level regarding whether the multiple tests were taken by the same person or multiple people, where a person's status may have changed between a first test and a second test (such as where the person had not acquired antibodies for a communicable illness before the first test but did before the second test), where a time threshold (such as a week) has passed between multiple tests, and so on. In various implementations, test kits registered to an identity may be required to be used within a time period, such as within three days of being registered, in order to prevent and/or reduce the possibility that people may register tests and then provide those tests to other people (and/or otherwise reduce the incentive for the person to attempt to invalidly associate a test result with his and/or another person's identity). Various configurations are possible and contemplated without departing from the scope of the present disclosure.

Other techniques may be used to prevent and/or reduce the possibility that a person will obtain multiple test kits and report that person's results for multiple different people (and/or otherwise reduce the incentive for the person to attempt to invalidly associate a test result with his and/or another person's identity). For example, test results may not be provided to a person until all tests for a family, household, and/or other relationship associated with the person's identity have been completed. In other examples, test results may be machine readable, encrypted, and/or otherwise stored in such a way that the test results are not readable by the person and must be accessed via the electronic device and/or other authorized device after reporting in order for the test results to be comprehensible.

In still other examples, correspondence between a test and an identity may instead be verified by obtaining video, images, and/or other data monitoring the person taking the test, such as via a home test kit. Such video, images, and/or other data may be collected and stored as proof of the correspondence, analyzed by one or more electronic devices and/or human monitors to look for suspicious activity and/or identify the test and/or the person (such as where a QR code and/or other identifier is captured from the test; where facial and/or other biometric recognition is used to identify the person in the video, images, and/or other data; where a remote witness attests that he witnessed the person take the test; and so on). Such video, images, and/or other data may be analyzed to verify chain of custody of the test. In other words, the video, images, and/or other data may be analyzed to verify that the same person unsealed and/or opened the test, performed the test on himself, sealed and/or otherwise completed the test, and provided the sealed and/or otherwise completed test sample for testing evaluation.

In yet other examples, correspondence between a test and an identity may instead be verified using an attestation of another person who witnessed the test, whether in person or remotely. For example, various people may be designated as authorized to witness tests. In another example, people may be authorized to witness tests upon completion of one or more background checks. In still other examples, lab and/or testing and/or medical service provider personnel may remotely witness people taking home tests. Various configurations are possible and contemplated without departing from the scope of the present disclosure.

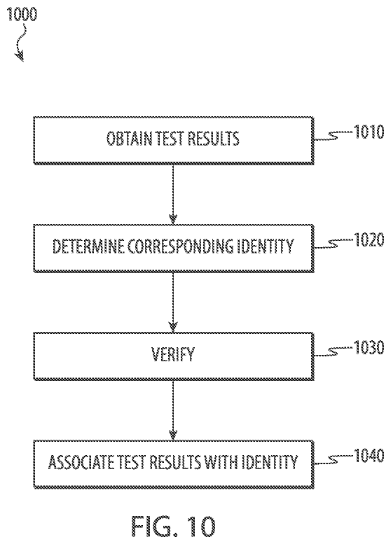

FIG. 10 is a flow chart illustrating a ninth example method 1000 for tracking and performing actions using health data. This method 1000 may be performed by the system 100 of FIG. 1.

At operation 1010, an electronic device, such as the identity system device 101 of FIG. 1, may obtain test results. At operation 1020, the electronic device may determine a corresponding identity of a person. For example, the electronic device may compare a digital representation of a biometric for the person with stored biometric data associated with identity information in order to determine the identity of the person that purportedly corresponds to the test results.

At operation 1030, the electronic device may verify correspondence of the test results to the identity. In other words, the electronic device may verify that the identity of the person that purportedly corresponds to the test results actually does correspond to the test results.

By way of illustration, the electronic device may compare biographic and/or other information stored in and/or otherwise associated with identity information for the person with biographic and/or other information associated with the test results. For example, such information may include first name, middle name or initial, last name, insurance information, address, gender, social security number and/or other identifier, and so on. When the electronic device determines that the biographic and/or other information stored in and/or otherwise associated with identity information for the person corresponds to that from the test results, the electronic device may determine that the test results are for the person and determine that the correspondence between the test results and the identity is verified.

In some implementations, the electronic device may determine that that one or more items of the biographic and/or other information stored in and/or otherwise associated with identity information for the person matches that from the test results in order to verify correspondence between the test results and the identity. In other implementations, the electronic device may determine that a threshold certainty level is met based on similarity between one or more items of the biographic and/or other information stored in and/or otherwise associated with identity information for the person and that from the test results in order to verify correspondence between the test results and the identity (such as where one lists a full middle name and the other lists a middle initial, where one lists a current address and the other lists an old address, and so on).

By way of another illustration, the electronic device may verify correspondence by verifying that the test results are not associated with health information that is inconsistent with health information stored in and/or otherwise associated with the identity information. For example, the test results may include data regarding age of the test subject, blood type of the test subject, DNA of the test subject, gender of the test subject, and/or other health information about the test subject that may have been obtained from a sample related to the test (such as a blood sample, a DNA sample, a mucus sample, and so on) and such data may be compared to health information stored in and/or otherwise associated with the identity information. In some implementations, the electronic device may verify correspondence by ensuring that the test results are not associated with an age inconsistent with that of the person, blood type inconsistent with that of the person, DNA inconsistent with that of the person, gender inconsistent with that of the person, and/or other health information inconsistent with that of the person. Such data may be gathered during testing. In implementations where the test is a home test kit, such data may be gathered by collecting test kits after test completion and later testing samples included in the collected test kits in order to obtain the data, whether routinely and/or in situations where additional verification is determined to be performed.

In yet another illustration, identities of people may be biometrically determined upon entry to a testing facility. In such a configuration, test results may be verified as corresponding to identities based upon the identities biometrically determined upon entry. Various configurations are possible and contemplated without departing from the scope of the present disclosure.

At operation 1040, the electronic device may associate the test results with the identity. This may include storing the test results in the identity information, storing the test results in a data store or enclave and/or a blockchain and/or other auditable record or ledger associated with the identity, and so on. For example, a blockchain and/or other auditable record or ledger may include one or more data blocks with one or more test result identifiers and/or one or more identifiers for and/or associated with the identity. The one or more test result identifiers and/or one or more identifiers for and/or associated with the identity may be used to associate the test results with identity information for the person, such as where the one or more test result identifiers and/or one or more identifiers for and/or associated with the identity are stored in the identity information and used to access the test results from the blockchain and/or other auditable record or ledger.

In various examples, this example method 1000 may be implemented as a group of interrelated software modules or components that perform various functions discussed herein. These software modules or components may be executed within a cloud network and/or by one or more computing devices, such as the identity system device 101 and/or the electronic device 102 of FIG. 1.

Although the example method 1000 is illustrated and described as including particular operations performed in a particular order, it is understood that this is an example. In various implementations, various orders of the same, similar, and/or different operations may be performed without departing from the scope of the present disclosure.

For example, the method 1000 illustrates obtaining the test results and determining the identity as separate, sequential operations. However, it is understood that this is an example. In other implementations, such operations may be performed in any order, including simultaneously, concurrent, and/or substantially simultaneously and/or concurrently. Various configurations are possible and contemplated without departing from the scope of the present disclosure.

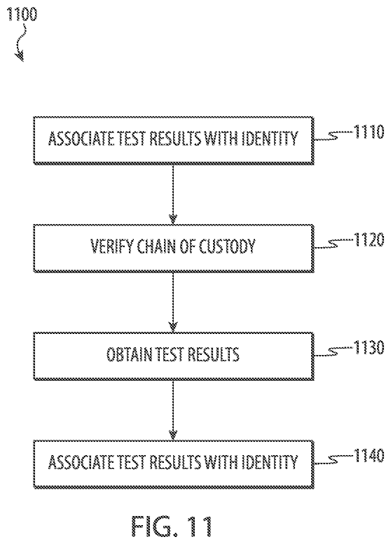

FIG. 11 is a flow chart illustrating a tenth example method 1100 for tracking and performing actions using health data. This method 1100 may be performed by the system 100 of FIG. 1.

At operation 1110, an electronic device, such as the identity system device 101 of FIG. 1, may associate a test with an identity. For example, a home test kit may be registered to an identity when purchased. By way of another example, an app or similar mechanism may be used to scan a Quick Read code or other bar code and/or identifier on the home test and/or serial number or other identifier on the test as well as one or more digital representations of one or more biometrics for the person, associating the particular home test with the identity of the person, which may deter the person from having another person take the test and/or falsely reporting the test result. In still other examples, a person has been prompted to take a home test and may be provided a home test already registered to his identity. An identity (and/or family, household, and/or other relationship associated with an identity) may be only able to be associated with the purchase of one home test kit in order to prevent and/or reduce the possibility that a person will obtain multiple test kits and report that person's results for multiple different people (and/or otherwise reduce the incentive for the person to attempt to invalidly associate a test result with his and/or another person's identity). Exceptions may be made for situations where a test has a high false negative rate, such as where a test with a high false negative rate may be overridden by a later test with sufficient verification that both tests were taken by the same person, where a less accurate test may be overridden by a later and more accurate test, where a home test may be overridden by a monitored test, where multiple tests are taken into account when determining a confidence level regarding whether the multiple tests were taken by the same person or multiple people, where a person's status may have changed between a first test and a second test (such as where the person had not acquired antibodies for a communicable illness before the first test but did before the second test), where a time threshold (such as a week) has passed between multiple tests, and so on. In various implementations, test kits registered to an identity may be required to be used within a time period, such as within three days of being registered, in order to prevent and/or reduce the possibility that people may register tests and then provide those tests to other people (and/or otherwise reduce the incentive for the person to attempt to invalidly associate a test result with his and/or another person's identity). Various configurations are possible and contemplated without departing from the scope of the present disclosure.

At operation 1120, the electronic device may verify chain of custody of the test. For example, chain of custody of the test may be verified by obtaining video, images, and/or other data monitoring the person taking the test, which may a home test kit. Such video, images, and/or other data may be collected and stored as proof of the chain of custody of the test, analyzed by one or more electronic devices and/or human monitors to look for suspicious activity and/or identify the test and/or the person (such as where a QR code and/or other identifier is captured from the test; where facial and/or other biometric recognition is used to identify the person in the video, images, and/or other data; where a remote witness attests that he witnessed the person take the test; and so on). Such video, images, and/or other data may be analyzed to verify that the same person unsealed and/or opened the test, performed the test on himself, sealed and/or otherwise completed the test, and provided the sealed and/or otherwise completed test sample for testing evaluation.

Alternatively and/or additionally, chain of custody of the test may be verified using an attestation of another person who witnessed the test, whether in person or remotely. For example, various people may be designated as authorized to witness tests. In another example, people may be authorized to witness tests upon completion of one or more background checks. In still other examples, lab and/or testing and/or medical service provider personnel may remotely witness people taking home tests. Various configurations are possible and contemplated without departing from the scope of the present disclosure.

At operation 1130, the electronic device may obtain test results for the test. At operation 1140, the electronic device may associate the test results with the identity. This may include storing the test results in the identity information, storing the test results in a data store or enclave and/or a blockchain and/or other auditable record or ledger associated with the identity, and so on. For example, a blockchain and/or other auditable record or ledger may include one or more data blocks with one or more test result identifiers and/or one or more identifiers for and/or associated with the identity. The one or more test result identifiers and/or one or more identifiers for and/or associated with the identity may be used to associate the test results with identity information for the person, such as where the one or more test result identifiers and/or one or more identifiers for and/or associated with the identity are stored in the identity information and used to access the test results from the blockchain and/or other auditable record or ledger. In some implementations, the test may auto report results and thus the auto reported test results may be associated with the identity to whom the test is registered.

In various examples, this example method 1100 may be implemented as a group of interrelated software modules or components that perform various functions discussed herein. These software modules or components may be executed within a cloud network and/or by one or more computing devices, such as the identity system device 101 and/or the electronic device 102 of FIG. 1.

Although the example method 1100 is illustrated and described as including particular operations performed in a particular order, it is understood that this is an example. In various implementations, various orders of the same, similar, and/or different operations may be performed without departing from the scope of the present disclosure.

For example, the method 1100 is illustrated and described as associating a test with an identity, verifying the chain of custody of the test, obtaining the test results, and associating the test results with the identity as separate, sequential operations. However, it is understood that this is an example. In other implementations, such operations may be performed in any order, including simultaneously, concurrently, and/or substantially simultaneously and/or concurrently. Various configurations are possible and contemplated without departing from the scope of the present disclosure.

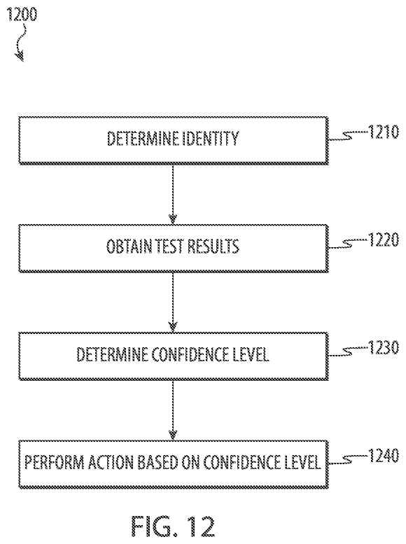

FIG. 12 is a flow chart illustrating an eleventh example method 1200 for tracking and performing actions using health data. This method 1200 may be performed by the system 100 of FIG. 1.

At operation 1210, an electronic device, such as the identity system device 101 of FIG. 1, may determine an identity of a person. For example, the electronic device may compare a digital representation of a biometric for the person with stored biometric data associated with identity information in order to determine the identity of the person. At operation 1220, the electronic device may obtain test results. At operation 1230, the electronic device may obtain a confidence level in the test results. At operation 1240, the electronic device may perform an action based on the confidence level. Such an action may include providing one or more attestations regarding the confidence level, controlling access based on the confidence level, allowing access when the confidence level is above a threshold, denying access when the confidence level is below the threshold, providing different levels of access for different confidence levels, storing the confidence level, providing information on how to change the confidence level, and so on.

For example, the confidence level may be determined based on whether the test is a home test kit, whether the test is a monitored test administered by medical personnel, whether or not the test is a home test registered to the person, whether or not biographic and/or other information included in and/or otherwise associated with identity information for the person corresponds to biographic and/or other information from the test, the degree to which biographic and/or other information included in and/or otherwise associated with identity information for the person corresponds to biographic and/or other information from the test, whether or not the test was witnessed, whether or not the test was witnessed in person, whether or not the test was witnessed remotely, whether or not video and/or images and/or other data monitoring the test is logged, whether or not video and/or images and/or other data monitoring the test is verified, whether or not a biological sample from the test was deposited, whether or not a biological sample from the test was verified as corresponding to the person, whether or not the test results are inconsistent with other test results, how much verification of the test and/or of the person was performed, a trustworthiness score of the person, the accuracy of the test, and/or any other factor that may indicate how accurate the test results are. The confidence level may then be used to perform an action, such as allowing the person access to an area (such as to an airport, restaurant, gym, flight, and so on) conditional to use of protective gear (such as a mask) and temperature or other health information verification when the confidence level is a low level, allow access conditional to use of protective gear without a temperature or other health information verification when the confidence level is a middle level, and access without use of protective gear and/or temperature or other health information verification when the access is a high level. Various configurations are possible and contemplated without departing from the scope of the present disclosure.

In various examples, this example method 1200 may be implemented as a group of interrelated software modules or components that perform various functions discussed herein. These software modules or components may be executed within a cloud network and/or by one or more computing devices, such as the identity system device 101 and/or the electronic device 102 of FIG. 1.

Although the example method 1200 is illustrated and described as including particular operations performed in a particular order, it is understood that this is an example. In various implementations, various orders of the same, similar, and/or different operations may be performed without departing from the scope of the present disclosure.

For example, the method 1200 is illustrated and described as obtaining the test results. However, it is understood that this is an example. In some implementations, the method 1200 may be used to determine a confidence level in one or more test results and perform one or more actions based thereon without obtaining the test results. Various configurations are possible and contemplated without departing from the scope of the present disclosure.

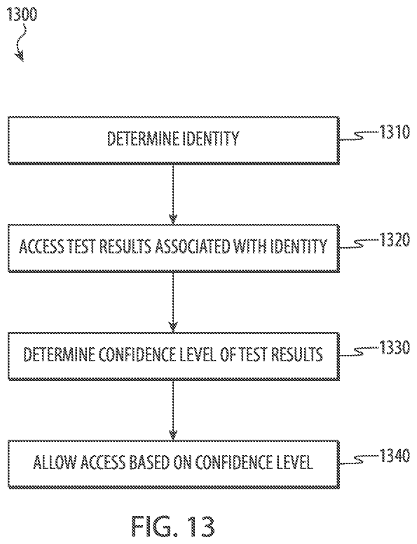

FIG. 13 is a flow chart illustrating a twelfth example method 1300 for tracking and performing actions using health data. This method 1300 may be performed by the system 100 of FIG. 1.

At operation 1310, an electronic device, such as the identity system device 101 of FIG. 1, may determine an identity of a person. For example, the electronic device may compare a digital representation of a biometric for the person with stored biometric data associated with identity information in order to determine the identity of the person. At operation 1320, the electronic device may access test results associated with the identity. At operation 1330, the electronic device may obtain a confidence level in the test results.

For example, the electronic device may determine the confidence level based on whether the test is a home test kit, whether the test is a monitored test administered by medical personnel, whether or not the test is a home test registered to the person, whether or not biographic and/or other information included in and/or otherwise associated with identity information for the person corresponds to biographic and/or other information from the test, the degree to which biographic and/or other information included in and/or otherwise associated with identity information for the person corresponds to biographic and/or other information from the test, whether or not the test was witnessed, whether or not the test was witnessed in person, whether or not the test was witnessed remotely, whether or not video and/or images and/or other data monitoring the test is logged, whether or not video and/or images and/or other data monitoring the test is verified, whether or not a biological sample from the test was deposited, whether or not a biological sample from the test was verified as corresponding to the person, whether or not the test results are inconsistent with other test results, how much verification of the test and/or of the person was performed, a trustworthiness score of the person, the accuracy of the test, and/or any other factor that may indicate how accurate the test results are. The confidence level may be on a numeric and/or other scale, such as a scale involving levels 1 through 5 where 1 is the lowest and 5 is the highest.

At operation 1340, the electronic device may allow access based on the confidence level. For example, the electronic device may allow different types of access to an area, such as to an airport, restaurant, gym, flight, and so on, based upon the confidence level associated with the test. By way of illustration, the electronic device may determine that the confidence level is 0 for no test, 1 for a home test, 2 for a remotely monitored home test, 3 for a home test where video of test administration was stored, 4 for a home test where a biological sample from the test was provided and verified as corresponding to the person, and 5 for a professionally administered and monitored test. The electronic device may then deny the person access when the confidence level is 0, allow the person access conditional to use of protective gear (such as a mask) and temperature or other health information verification when the confidence level is 1, allow access other than to congregating areas conditional to use of protective gear without a temperature or other health information verification when the confidence level is 2, allow access other than to congregating areas without use of protective gear and/or temperature or other health information verification when the access is 3, allow access even to congregating areas conditional to use of protective gear without a temperature or other health information verification when the confidence level is 4, and allow access even to congregating areas without use of protective gear and/or temperature or other health information verification when the access is 5. Various configurations are possible and contemplated without departing from the scope of the present disclosure.

In various examples, this example method 1300 may be implemented as a group of interrelated software modules or components that perform various functions discussed herein. These software modules or components may be executed within a cloud network and/or by one or more computing devices, such as the identity system device 101 and/or the electronic device 102 of FIG. 1.

Although the example method 1300 is illustrated and described as including particular operations performed in a particular order, it is understood that this is an example. In various implementations, various orders of the same, similar, and/or different operations may be performed without departing from the scope of the present disclosure.

For example, the method 1300 is illustrated and described as allowing access based on the confidence level. However, it is understood that this is an example. In other implementations, other actions may be performed. For example, in some implementations, access may be denied based on the confidence level. Various configurations are possible and contemplated without departing from the scope of the present disclosure.

The techniques described herein have been described in the context of specific examples. However, it is understood that these are examples and that the techniques described herein may be used in other contexts without departing from the scope of the present disclosure.

By way of example, in various implementations, the techniques herein may be used to inspire customer confidence, such as for ride sharing, the hospitality and dining industries, and so on. By way of a first illustration, the techniques discussed herein may be used to provide an attestation that a given vehicle is not a communicable illness risk based on having cleared all of the drivers and all riders and/or other people who have come into contact with the vehicle. By way of a second illustration, the techniques discussed herein may be used to provide an attestation that a given room is not a communicable illness risk based on having cleared all staff and all lodgers and/or other people who have come into contact with the room. By way of a third illustration, the techniques discussed herein may be used to provide an attestation that a given restaurant or store is not a communicable illness risk based on having cleared all staff and all customers who have come into contact with the store, as well as people and/or goods involved in the supply chain for the restaurant or store. Various configurations are possible and contemplated without departing from the scope of the present disclosure.

In a particular example, the techniques discussed herein may be used to implement a safe basketball game and/or similar package. By way of illustration, all of the ticket holders, employees, rooms and/or other areas, restaurants, transportation, and so on may be cleared for a package that allows a group of ticket holders to stay at a cleared hotel, eat at a cleared restaurant, and ride in cleared transportation to a set (such as three, four, and so on) of cleared basketball games. In this way, the ticketholders and other people may be able to stay at a hotel, eat at a restaurant, ride transportation, and attend basketball games without risk of contracting a communicable illness and/or spreading such a communicable illness themselves, even in the midst of high risk conditions for communicable illnesses. Various configurations are possible and contemplated without departing from the scope of the present disclosure.

Further, although the above discusses use of these attestations in the context of consumer confidence, in other implementations, such attestations may be used in the context of insurance underwriting for a business, debt rating for a business, and so on. Such contexts may take such attestations into account when performing insurance underwriting, debt rating, and so on, and/or such contexts may consider the fact that a business or other entity participates in such evaluations as evidence of the business's or other entity's resilience. Various configurations are possible and contemplated without departing from the scope of the present disclosure.

By way of another example, the techniques of the present disclosure may be used to provide an attestation that the chain of custody for goods, such as delivered goods, is free of a risk of a communicable illness. This may be performed by clearing everyone involved in the chain of custody for the goods, and/or people with whom those people have come into contact. This may be used in the context of grocery or meal delivery, package and/or other mail delivery, and so on. Various configurations are possible and contemplated without departing from the scope of the present disclosure.

In various examples, the techniques described herein may be used to provide incentives for people who opt-in and share health information. For example, people who are determined to have factors that might make them risks of communicating communicable illnesses might be offered special services in exchange for sharing health information, such as free contactless delivery of food to their doorstep with text notification on arrival in exchange for being willing to share information that delivery personnel may want to avoid direct contact with them. By way of another example, people who are willing to share health information may be provided free or discounted travel insurance upon ticket purchase, the free or discounted travel insurance being provided in exchange for the possibility that their shared health information causes a flight or other ticketed event to be missed or a reservation to be denied. In other examples, seat or status upgrades may be provided, or mileage or loyalty point multipliers may be applied during a time period that health information is shared. In yet other examples, free or discounted fast lane access to amusement park rides may be provided for sharing health information. In still other examples, prioritized access may be provided to those who have agreed to share health information over those who have not agreed. Various configurations are possible and contemplated without departing from the scope of the present disclosure.

In a number of examples, incentives may be provided for linking wearable health data with identity information so that communicable illness risk decisions may be made using a recent history of health data instead of a single instance of data taken at a kiosk or other station. For example, season ticket holders of a basketball team may be provided with free and/or discounted identity system membership, with a free fitness monitor and/or other wearable health device, and so on in exchange for sharing wearable health data, wearing the fitness monitor and/or other wearable health device to games, and so on.

In yet another example, the techniques described herein may be used to implement a system where health care employees (such as doctors, nurses, lab technicians, and so on) check in and/or check out for work, such as by providing one or more digital representations of one or more biometrics. Such a system may correlate to health information for the health care employees, correlate to people that the health care employees have come into contact with, and so on. This may be used for a variety of purposes, such as to screen for access when checking into work based on risk of communicable illness, determining whether or not a health care employee may need to be isolated upon checking out from work, making informed staffing and/or work assignment decisions to minimize communicable illness transmission risk for health care employees and/or patients, and so on. Various configurations are possible and contemplated without departing from the scope of the present disclosure.

In still another example, the techniques of the present disclosure may be used in the context of public transportation and/or other transportation. By way of illustration, the techniques discussed herein may be used to provide biometric and/or other heath checks for employees, passengers, and so on. Passenger and/or employee health information (and/or the health information of other people) may be used to determine seating assignments, to segregate one population from another (such as vulnerable people from at risk people and so on), to assign people to particular vehicles, and so on. Various configurations are possible and contemplated without departing from the scope of the present disclosure.

Various techniques may be used in connection with the present disclosure to protect stored health information, to avoid storing health information where not necessary, to comply with privacy regulations regarding health information (such as HIPAA), and so on. For example, in some implementations health information may not be stored in identity information and an identity system may instead act as a switch to exchange data held by others when appropriately authorized. In another example, the identity system may collect the health information, which may not be subject to particular regulations. In still other examples, health information may be obtained from private entities who do not accept health insurance and may thus not be subject to particular regulations. In yet other examples, people may provide one or more waivers related to one or more regulations to an identity system that the identity system may then use to obtain the health information in compliance with the regulations. In still other examples, health care providers may obtain permission from individuals to share information with an identity system and may then send data in bulk for those who have permitted the information to be shared. Various configurations are possible and contemplated without departing from the scope of the present disclosure.

In various examples of implementations that use techniques of the present disclosure, a telemedicine/telehealth medical service provider system may receive a digital representation of a biometric from a new patient. The telemedicine/telehealth medical service provider system may provide the received digital representation of the biometric to an identity system and may receive in response a verification of the new patient's identity, identity information about the new patient, attestations about the new patient, health information for the new patient, and so on. In this way, the telemedicine/telehealth medical service provider may be able to securely and accurately onboard new patients remotely. Various configurations are possible and contemplated without departing from the scope of the present disclosure.

In some examples of implementations that use techniques of the present disclosure, healthcare workers may provide a digital representation of a biometric to a device upon returning to work in order to attest that the healthcare worker is healthy to work. The device may provide the received digital representation of the biometric to an identity system and may receive in response one or more attestations regarding the health of the healthcare worker and/or that the healthcare worker is healthy to work. Various configurations are possible and contemplated without departing from the scope of the present disclosure.

In a number of examples of implementations that use techniques of the present disclosure, a ballpark or other event venue may perform health screenings on people entering. Such health screenings may involve taking temperatures, nasal swabs, or other time consuming and/or burdensome procedures. However, people may be able to provide a digital representation of a biometric that may be used via an identity system to verify that the person has recently been tested. Such stored recent health information may be relied upon and the person may be allowed to bypass the health screening. In this way, the people may provide the digital representation of the biometric to attest to the ballpark or other event venue that they are healthy and do not require health screening. Various configurations are possible and contemplated without departing from the scope of the present disclosure.

In various examples of implementations that use techniques of the present disclosure, a station outside a hotel or other area where people congregate may enable people to provide a digital representation of a biometric that may be used to obtain health information for the people and/or attestations based thereon. The station may evaluate the health information and/or attestation(s) against a current set of requirements set by the hotel or other area in order to determine whether or not to allow the people to enter. Various configurations are possible and contemplated without departing from the scope of the present disclosure.

In some examples of implementations that use techniques of the present disclosure, health and/or other information monitored and/or tracked regarding people who have utilized a room, rental vehicle, object, and so on may be recorded in a blockchain and/or other auditable record or ledger. A person contemplating using the room, rental vehicle, object, and so on may be able to access the blockchain to verify that all of the previous people had been healthy. Alternatively, an offeror of the room, rental vehicle, object, and so on may use the blockchain to attest to the person that all of the previous people had been healthy. Various configurations are possible and contemplated without departing from the scope of the present disclosure.

In various examples of implementations that use techniques of the present disclosure, test results (such as results of an antibody test, a temperature test, and/or any other kind of medical and/or health test) may be recorded in a blockchain and/or other auditable record or ledger. The blockchain and/or other auditable record or ledger may include one or more test result identifiers and/or one or more identifiers for a person associated with the test result. The one or more test result identifiers and/or one or more identifiers for the person may be used to associate the test results with identity information for the person, such as where the one or more test result identifiers and/or one or more identifiers for the person are stored in the identity information and used to access the test results from the blockchain and/or other auditable record or ledger. In some examples, the test results in the blockchain and/or other auditable record or ledger may be machine readable, encrypted, and/or otherwise stored in such a way that the test results are not readable by the person from the blockchain and/or other auditable record or ledger and must be accessed via the identity information in order for the test results to be comprehensible. This may prevent a person from learning of adverse test results stored in the blockchain and/or other auditable record or ledger and then not associating those adverse test results with the identity information.

In a number of examples of implementations of techniques of the present disclosure, a person requesting a delivery transaction may provide a digital representation of a biometric that may be used via an identity system to access health information for the person and determine that the person is at risk of having a communicable illness. A worker fulfilling the delivery transaction may be notified of this risk so that the worker may perform the delivery in a contactless fashion, require contactless payment, and/or otherwise maintain an appropriate distance from the person and/or anything that person has come in contact with. Various configurations are possible and contemplated without departing from the scope of the present disclosure.

In various examples of implementations of techniques of the present disclosure, a wearable device associated with a person may cooperate with the systems discussed herein. For example, a person may have a wearable device that monitors information about the person's body, such as pulse rate, temperature, and so on. When the person provides a digital representation of a biometric to a device that communicates the digital representation of the biometric to an identity system, the wearable device may be configured to communicate the monitored information to the device and/or the identity system for storage, evaluation, determining trends, and/or other purposes. In this way, a larger amount of health information may be made available to the identity system and/or devices that communicate therewith for purposes of storage, evaluation, determining trends, and so on. Various configurations are possible and contemplated without departing from the scope of the present disclosure.

In various implementations, a system for tracking and performing actions using health data may include at least one non-transitory storage medium that stores instructions and at least one processor. The at least one processor may execute the instructions to obtain a digital representation of a biometric for a person, determine an identity for the person using the digital representation of the biometric, determine an access account identifier stored in identity information associated with the identity, use the access account identifier to determine whether or not the person has an access permission, evaluate heath information for the person, and determine whether to allow the person access based on the access permission and the health information.

In some examples, the at least one processor may obtain the health information for the person using a sensor. In a number of such examples, the at least one processor may obtain the digital representation of the biometric for the person using the sensor.

In various examples, the at least one processor may obtain the health information from a data store associated with the identity information. In some such examples, the health information may include a recently monitored temperature for the person.

In a number of examples, the health information may include a currently monitored temperature for the person. In various examples, the at least one processor may be operable to receive the health information and store the health information in association with the identity information.

In some implementations, a system for tracking and performing actions using health data may include at least one non-transitory storage medium that stores instructions and at least one processor. The at least one processor may execute the instructions to obtain a digital representation of a biometric for a person, determine an identity for the person using the digital representation of the biometric, retrieve health information for the person stored in association with identity information associated with the identity, evaluate the health information, and provide an attestation based on the health information.

In various examples, the digital representation of the biometric may be a first digital representation of the biometric and the health information may include data previously received with a second digital representation of the biometric by the at least one processor.

In a number of examples, the attestation may indicate that the person has a particular vaccination or that results of an antibody test evidence that the person has had a particular communicable illness and recovered. In some examples, the attestation may indicate that a recently monitored temperature for the person is a normal temperature. In a number of examples, the attestation may indicate that a recently monitored temperature for the person is an abnormal temperature. In various examples, the attestation may indicate that the person is not a significant risk of having a particular communicable illness. In some examples, the at least one processor may be operable to receive at least one medical record associated with the identity, verify the at least one medical record, and store the at least one medical record in association with the identity information.

In a number of implementations, a system for tracking and performing actions using health data may include at least one non-transitory storage medium that stores instructions and at least one processor. The at least one processor may execute the instructions to obtain a digital representation of a biometric for a person, determine an identity for the person using the digital representation of the biometric, evaluate health information for the person stored in association with identity information associated with the identity, and route the person based on the health information.

In some examples, the at least one processor may route the person by assigning the person a seat.

In various examples, the at least one processor may evaluate the health information to determine that the person encountered an infected person. In some such examples, the at least one processor may determine whether the person is tested for a communicable illness after encountering the infected person. In various such examples, the at least one processor may determine whether the person is vaccinated for a communicable illness after encountering the infected person or results of an antibody test indicate that the person has recovered from the communicable illness.

In a number of examples, the at least one processor may route the person in a first manner if the person is not at risk of having a communicable illness and a second manner if the person cannot be determined to not be at risk of having the communicable illness.

Although the above illustrates and describes a number of embodiments, it is understood that these are examples. In various implementations, various techniques of individual embodiments may be combined without departing from the scope of the present disclosure.

As described above and illustrated in the accompanying figures, the present disclosure relates to tracking and performing actions using health data. The system is operable to use digital representations of biometrics to control access to identity information for people stored in an identification system. The system uses the stored identity information to track, evaluate, and/or correlate current and/or previously monitored health information for the people to perform one or more of a variety of actions. Such actions may include determining whether or not to allow the person access, providing attestations about a person's health information, routing the person based on one or more evaluations of the health information, and so on.

Although the above illustrates and describes a number of embodiments, it is understood that these are examples. In various implementations, various techniques of individual embodiments may be combined without departing from the scope of the present disclosure.

The present disclosure recognizes that biometric and/or other personal data is owned by the person from whom such biometric and/or other personal data is derived. This data can be used to the benefit of those people. For example, biometric data may be used to conveniently and reliably identify and/or authenticate the identity of people, access securely stored financial and/or other information associated with the biometric data, and so on. This may allow people to avoid repeatedly providing physical identification and/or other information.

The present disclosure further recognizes that the entities who collect, analyze, store, and/or otherwise use such biometric and/or other personal data should comply with well-established privacy policies and/or privacy practices. Particularly, such entities should implement and consistently use privacy policies and practices that are generally recognized as meeting or exceeding industry or governmental requirements for maintaining security and privately maintaining biometric and/or other personal data, including the use of encryption and security methods that meets or exceeds industry or government standards. For example, biometric and/or other personal data should be collected for legitimate and reasonable uses and not shared or sold outside of those legitimate uses. Further, such collection should occur only after receiving the informed consent. Additionally, such entities should take any needed steps for safeguarding and securing access to such biometric and/or other personal data and ensuring that others with access to the biometric and/or other personal data adhere to the same privacy policies and practices. Further, such entities should certify their adherence to widely accepted privacy policies and practices by subjecting themselves to appropriate third party evaluation.

Additionally, the present disclosure recognizes that people may block the use of, storage of, and/or access to biometric and/or other personal data. Entities who typically collect, analyze, store, and/or otherwise use such biometric and/or other personal data should implement and consistently prevent any collection, analysis, storage, and/or other use of any biometric and/or other personal data blocked by the person from whom such biometric and/or other personal data is derived.

In the present disclosure, the methods disclosed may be implemented as sets of instructions or software readable by a device. Further, it is understood that the specific order or hierarchy of steps in the methods disclosed are examples of sample approaches. In other embodiments, the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A non-transitory machine-readable medium includes any mechanism for storing information in a form (e.g., software, processing application) readable by a machine (e.g., a computer). The non-transitory machine-readable medium may take the form of, but is not limited to, a magnetic storage medium (e.g., floppy diskette, video cassette, and so on); optical storage medium (e.g., CD-ROM); magneto-optical storage medium; read only memory (ROM); random access memory (RAM); erasable programmable memory (e.g., EPROM and EEPROM); flash memory; and so on.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A system for tracking and performing actions using health data, comprising:
at least one non-transitory storage medium that stores instructions; and
at least one processor that executes the instructions to:
obtain a digital representation of a biometric for a person;
determine an identity for the person using the digital representation of the biometric;
determine an access account identifier stored in identity information associated with the identity;
use the access account identifier to determine whether or not the person has an access permission;
evaluate heath information for the person; and
determine whether to allow the person access based on the access permission and the health information.

2. The system of claim 1, wherein the at least one processor obtains the health information for the person using a sensor.

3. The system of claim 2, wherein the at least one processor obtains the digital representation of the biometric for the person using the sensor.

4. The system of claim 1, wherein the at least one processor obtains the health information from a data store associated with the identity information.

5. The system of claim 4, wherein the health information includes a recently monitored temperature for the person.

6. The system of claim 1, wherein the health information includes a currently monitored temperature for the person.

7. The system of claim 1, wherein the at least one processor is operable to receive the health information and store the health information in association with the identity information.

8. A system for tracking and performing actions using health data, comprising:
at least one non-transitory storage medium that stores instructions; and
at least one processor that executes the instructions to:
obtain a digital representation of a biometric for a person;
determine an identity for the person using the digital representation of the biometric;
retrieve health information for the person stored in association with identity information associated with the identity;
evaluate the health information; and
provide an attestation based on the health information.

9. The system of claim 8, wherein:
the digital representation of the biometric is a first digital representation of the biometric; and
the health information includes data previously received with a second digital representation of the biometric by the at least one processor.

10. The system of claim 8, wherein the attestation indicates that the person has a particular vaccination or that results of an antibody test evidence that the person has had a particular communicable illness and recovered.

11. The system of claim 8, wherein the attestation indicates that a recently monitored temperature for the person is a normal temperature.

12. The system of claim 8, wherein the attestation indicates that a recently monitored temperature for the person is an abnormal temperature.

13. The system of claim 8, wherein the attestation indicates that the person is not a significant risk of having a particular communicable illness.

14. The system of claim 8, wherein the at least one processor is operable to:
receive at least one medical record associated with the identity;
verify the at least one medical record; and
store the at least one medical record in association with the identity information.

15. A system for tracking and performing actions using health data, comprising:
at least one non-transitory storage medium that stores instructions; and
at least one processor that executes the instructions to:
obtain a digital representation of a biometric for a person;
determine an identity for the person using the digital representation of the biometric;
evaluate health information for the person stored in association with identity information associated with the identity; and
route the person based on the health information.

16. The system of claim 15, wherein the at least one processor routes the person by assigning the person a seat.

17. The system of claim 15, wherein the at least one processor evaluates the health information to determine that the person encountered an infected person.

18. The system of claim 17, wherein the at least one processor determines whether the person is tested for a communicable illness after encountering the infected person.

19. The system of claim 17, wherein the at least one processor determines whether the person is vaccinated for a communicable illness after encountering the infected person or that results of an antibody test evidence that the person has had the communicable illness and recovered.

20. The system of claim 15, wherein the at least one processor routes the person in:
a first manner if the person is not at risk of having a communicable illness; and
a second manner if the person cannot be determined to not be at risk of having the communicable illness.
